(19) United States
(12) Patent Application Publication
(10) Pub. No.: US 2024/0409501 A1
(43) Pub. Date: Dec. 12, 2024

(54) CYRSTALLINE FORMS OF RIP1 INHIBITOR

(71) Applicant: SIRONAX LTD., Grand Cayman (KY)

(72) Inventors: Yaning SU, Beijing (CN); Shaoqiang HUANG, Beijing (CN); Zhiyuan ZHANG, Beijing (CN); Wenfeng XUE, Suzhou (CN); Jun XU, Suzhou (CN)

(21) Appl. No.: 18/699,303

(22) PCT Filed: Oct. 21, 2022
(86) PCT No.: PCT/CN2022/126631

§ 371 Date: Apr. 8, 2024

(30) Foreign Application Priority Data
Oct. 22, 2021 (WO) PCT/CN2021/125895

(51) Int. Cl. C07C 259/06; A61K 31/16

(52) U.S. Cl. CPC C07C 259/06; A61K 31/16

(57) ABSTRACT

Provide crystalline forms of N-(3,5-difluorobenzyl)-N-hydroxy-2,2-dimethylbutanamide, methods of preparing and characterizing the crystalline forms, and methods of using the crystalline forms to treat various diseases or conditions, e.g., those mediated by RIP1.

### RELATED APPLICATIONS

This application claims priority to International Application No. PCT/CN2021/125895, filed on Oct. 22, 2021, the content of which is incorporated herein by reference in its entirety.

### FIELD OF THE DISCLOSURE

The present disclosure relates to solid forms, e.g., crystalline forms, of N-(3,5-difluorobenzyl)-N-hydroxy-2,2-dimethylbutanamide, methods of preparing and characterizing the crystalline forms, and methods of using the crystalline forms to treat diseases or conditions, e.g., those mediated by receptor-interacting protein 1 (RIP1).

### BACKGROUND OF THE DISCLOSURE

Necroptosis, an important form of programmed cell death, is a highly regulated caspase-independent type of cell death that plays a critical role in many necrotic cell diseases, manifested in various pathological forms of cell death, including ischemic brain injury, neurodegenerative diseases, viral infections, and peripheral autoimmune diseases. (Dunai, et al., December 2011, Pathol. Oncol. Res.: POR 17 (4): 791-800. J. Med. Chem. 2020, 63, 4, 1490-1510. Nature Reviews Drug Discovery, 19, 553-571(2020)). Tumor necrosis factor alpha (TNF-α)-induced NF-κB activation plays a central role in the immune system and inflammatory responses.

RIP1 is a multi-functional signal transducer involved in mediating nuclear factor κB (NF-κB) activation, apoptosis, and necroptosis. The kinase activity of RIP1 is critically involved in mediating necroptosis, a caspase-independent pathway of necrotic cell death. (Holler et al. Nat Immunol 2000; 1: 489-495; Degterev et al. Nat Chem Biol 2008; 4: 313-321). RIP1 can contribute to D-1 immunotherapy resistance (e.g., Manguso et al., 2017 Nature 547, 413-418) and can act as a checkpoint kinase governing tumor immunity (e.g., Wang et al, Cancer Cell 34, 757-774, Nov. 12, 2018). RIP1 has emerged as a promising therapeutic target for the treatment of a wide range of human neurodegenerative, autoimmune, and inflammatory diseases, such as psoriasis, rheumatoid arthritis, and ulcerative colitis (Pharmacol. Res. Perspect. 2017, 5, e00365, PNAS May 14, 2019, 116 (20) 9714-9722), as well for central nervous system (CNS) indications such as amyotrophic lateral sclerosis (ALS) and Alzheimer's disease. (Nat. Rev. Neurosci. 2019, 20, 19-33).

Certain compounds for modulating necrosis or necroptosis are disclosed in U.S. Pat. Nos. 9,974,762, 10,092,529, 6,756,394, 8,278,344, U.S. Patent Publication No. 20120122889, U.S. Patent Publication No. 20090099242, U.S. Patent Publication No. 20100317701, U.S. Patent Publication No. 20110144169, U.S. Patent Publication No. 20030083386, U.S. Patent Publication No. 201200309795, WO2009023272, WO2010075290, WO2010075561, WO2012125544, WO 2020/103884, and WO2020103859.

It is desirable to obtain various solid forms of RIP1 inhibitors, such as crystalline forms of the inhibitors, that are suitable for therapeutic uses and manufacturing processes.

### SUMMARY OF THE DISCLOSURE

One aspect of this disclosure provides solid state forms, such as crystalline forms, of N-(3,5-difluorobenzyl)-N-hydroxy-2,2-dimethylbutanamide, as shown in Formula I.

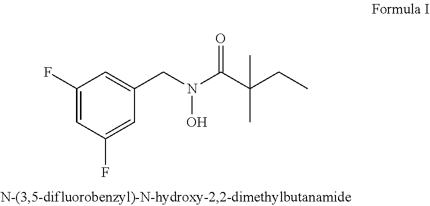 [SMILES: CCC(C)(C)C(=O)N(O)Cc1cc(F)cc(F)c1]

In some embodiments, the present disclosure provides crystalline Type A of N-(3,5-difluorobenzyl)-N-hydroxy-2,2-dimethylbutanamide, e.g., in substantially pure form.

In some embodiments, the present disclosure provides crystalline Type C of N-(3,5-difluorobenzyl)-N-hydroxy-2,2-dimethylbutanamide, e.g., in substantially pure form.

In some embodiments, the disclosure provides pharmaceutical compositions comprising a substantially pure crystalline form (e.g., Type A or Type C) of N-(3,5-difluorobenzyl)-N-hydroxy-2,2-dimethylbutanamide and a pharmaceutically acceptable carrier. In some embodiments, the pharmaceutical compositions may further comprise an additional active pharmaceutical agent.

Another aspect of the disclosure provides methods of treating a disease or condition, comprising administering to a subject in need thereof, a therapeutically effective amount of a substantially pure crystalline form (e.g., Type A or Type C) of N-(3,5-difluorobenzyl)-N-hydroxy-2,2-dimethylbutanamide or a pharmaceutical composition thereof, wherein the disease or condition is selected from an inflammatory disease, an immune disease (e.g., an autoimmune disease), an allergic disease, transplant rejection, a necrotic cell disease, a neurodegenerative disease, a CNS disease, an ocular disease, an infectious disease, a malignancy, ulcerative colitis, Crohn's disease, psoriasis, rheumatoid arthritis, ALS, Alzheimer's disease, and a viral infection.

A further aspect of the disclosure provides methods of treating a disease or condition mediated by RIP1, comprising administering to a subject in need thereof, a therapeutically effective amount of a substantially pure crystalline form (e.g., Type A or Type C) of N-(3,5-difluorobenzyl)-N-hydroxy-2,2-dimethylbutanamide or a pharmaceutical composition thereof.

In some embodiments, the methods of treatment comprise administration of an additional active pharmaceutical agent to the subject in need thereof, either in the same pharmaceutical composition as a substantially pure crystalline form (e.g., Type A or Type C) of N-(3,5-difluorobenzyl)-N-hydroxy-2,2-dimethylbutanamide, or in a separate composition. When administered as a separate dosage form, the additional therapeutic agent may be administered prior to, at the same time as, or following administration of the crystalline form.

Also disclosed herein are methods of mediating, e.g., inhibiting, RIP1, comprising contacting a RIP1 protein or a fragment thereof with a substantially pure crystalline form (e.g., Type A or Type C) of N-(3,5-difluorobenzyl)-N-hydroxy-2,2-dimethylbutanamide or a pharmaceutical composition thereof.

### BRIEF DESCRIPTION OF THE DRAWINGS

In certain drawings showing multiple graphs, the legend in the boxes in the top right corner of the drawings indicate, from top to bottom, the identification numbers of the materials used for the respective graphs in the drawings. In certain drawings, the individual graphs are also separately annotated with the identification numbers of the materials used for the respective graphs.

FIG. 1 shows an XRPD pattern of crystalline Type A of N-(3,5-difluorobenzyl)-N-hydroxy-2,2-dimethylbutanamide (Experiment ID 817506-48-A, also referred to as “Type A ref” or “Type A_817506-48-A” herein).

FIG. 2 shows a DSC thermogram (bottom) and a TGA graph (top) of crystalline Type A of N-(3,5-difluorobenzyl)-N-hydroxy-2,2-dimethylbutanamide (Experiment ID 817506-48-A).

FIG. 3 shows proton NMR of crystalline Type A of N-(3,5-difluorobenzyl)-N-hydroxy-2,2-dimethylbutanamide (Experiment ID 817506-48-A) using DMSO-d6 as a solvent.

FIG. 4 shows a PLM image of crystalline Type A of N-(3,5-difluorobenzyl)-N-hydroxy-2,2-dimethylbutanamide (Experiment ID 817506-48-A).

FIG. 5A shows an XRPD pattern of crystalline Type A of N-(3,5-difluorobenzyl)-N-hydroxy-2,2-dimethylbutanamide (unmicronized); FIG. 5B shows a DSC thermogram of crystalline Type A of N-(3,5-difluorobenzyl)-N-hydroxy-2,2-dimethylbutanamide (unmicronized), with the first peak showing melting of the compound and the second peak showing decomposition of the compound.

FIG. 6A shows an XRPD pattern of crystalline Type A of N-(3,5-difluorobenzyl)-N-hydroxy-2,2-dimethylbutanamide (micronized); FIG. 6B shows a DSC thermogram of crystalline Type A of N-(3,5-difluorobenzyl)-N-hydroxy-2,2-dimethylbutanamide (micronized), with the first peak showing melting of the compound and the second peak showing decomposition of the compound. The particle size (D90) of the micronized sample was about 15 μm.

FIG. 7 shows XRPD patterns of two samples of crystalline Type A of N-(3,5-difluorobenzyl)-N-hydroxy-2,2-dimethylbutanamide obtained from an anti-solvent addition method (Experiment IDs 810048-19-B1 and 810048-19-B2), Type A Ref. (Experiment ID 817506-48-A), and Impurity B ref.

FIG. 8 shows a DSC thermogram of crystalline Type A of N-(3,5-difluorobenzyl)-N-hydroxy-2,2-dimethylbutanamide obtained from an anti-solvent addition method (Experiment ID 810048-19-B2).

FIG. 9 shows XRPD patterns of five samples of crystalline Type A of N-(3,5-difluorobenzyl)-N-hydroxy-2,2-dimethylbutanamide obtained from a solid vapor diffusion method (Experiment IDs 810055-02-A1, 810055-02-A2, 810055-02-A3, 810055-02-A4, and 810055-02-A5), Type A Ref. (Experiment ID 817506-48-A), and Impurity B ref.

FIG. 10 shows DSC thermograms of two samples of crystalline Type A of N-(3,5-difluorobenzyl)-N-hydroxy-2,2-dimethylbutanamide obtained from a solid vapor diffusion method (Experiment IDs 810055-02-A1 (top) and 810055-02-A3 (bottom)).

FIG. 11 shows XRPD patterns of four samples of crystalline Type A of N-(3,5-difluorobenzyl)-N-hydroxy-2,2-dimethylbutanamide obtained from a slurry at room temperature method (Experiment IDs 810055-03-A1, 810055-03-A2, 810055-03-A3, and 810055-03-A4), Type A Ref. (Experiment ID 817506-48-A), and Impurity B ref.

FIG. 12 shows XRPD patterns of four samples of crystalline Type A of N-(3,5-difluorobenzyl)-N-hydroxy-2,2-dimethylbutanamide obtained from a slurry at room temperature method (Experiment IDs 810055-03-A5, 810055-03-A6, 810055-03-A7, and 810055-03-A8), Type A Ref. (Experiment ID 817506-48-A), and Impurity B ref.

FIG. 13 shows XRPD patterns of three samples of crystalline Type A of N-(3,5-difluorobenzyl)-N-hydroxy-2,2-dimethylbutanamide obtained from a slurry at room temperature method (Experiment IDs 810055-03-A9, 810055-03-A10, and 810055-03-A11), Type A Ref. (Experiment ID 817506-48-A), and Impurity B ref.

FIG. 14 shows DSC thermograms of three samples of crystalline Type A of N-(3,5-difluorobenzyl)-N-hydroxy-2,2-dimethylbutanamide obtained from a slurry at room temperature method (Experiment IDs 810055-03-A1 (top), 810055-03-A6 (middle), and 810055-03-A9 (bottom)).

FIG. 15 shows XRPD patterns of three samples of crystalline Type A of N-(3,5-difluorobenzyl)-N-hydroxy-2,2-dimethylbutanamide obtained from a slurry at 50° C. method (Experiment IDs 810055-04-A1, 810055-04-A2, and 810055-04-A3), Type A Ref. (Experiment ID 817506-48-A), and Impurity B ref.

FIG. 16 shows XRPD patterns of three samples of crystalline Type A of N-(3,5-difluorobenzyl)-N-hydroxy-2,2-dimethylbutanamide obtained from a slurry at 50° C. method (Experiment IDs 810055-04-A5, 810055-04-A6, and 810055-04-A7), Type A Ref. (Experiment ID 817506-48-A), and Impurity B ref.

FIG. 17 shows DSC thermograms of two samples of crystalline Type A of N-(3,5-difluorobenzyl)-N-hydroxy-2,2-dimethylbutanamide obtained from a slurry at 50° C. method (Experiment IDs 810055-04-A3 (top) and 810055-04-A6 (bottom)).

FIG. 18 shows XRPD patterns of three samples of crystalline Type A of N-(3,5-difluorobenzyl)-N-hydroxy-2,2-dimethylbutanamide obtained from a slow evaporation method (Experiment IDs 810055-05-A1, 810055-05-A2, and 810048-19-B3), Type A Ref. (Experiment ID 817506-48-A), and Impurity B ref.

FIG. 19 shows DSC thermograms of three samples of crystalline Type A of N-(3,5-difluorobenzyl)-N-hydroxy-2,2-dimethylbutanamide obtained from a slow evaporation method (Experiment IDs 810055-05-A1, 810055-05-A2, and 810048-19-B3).

FIG. 20 shows XRPD patterns of three samples of crystalline Type A of N-(3,5-difluorobenzyl)-N-hydroxy-2,2-dimethylbutanamide obtained from a slow cooling method (Experiment IDs 810055-06-A1 (top), 810055-06-A2 (middle), and 810048-19-B5 (bottom)), Type A Ref (Experiment ID 817506-48-A), and Impurity B ref FIG. 21 shows DSC thermograms of two samples of crystalline Type A of N-(3,5-difluorobenzyl)-N-hydroxy-2,2-dimethylbutanamide obtained from a slow cooling method (Experiment IDs 810055-06-A2 (top) and 810048-19-B5 (bottom)).

FIG. 22 shows XRPD patterns of four samples of crystalline Type A of N-(3,5-difluorobenzyl)-N-hydroxy-2,2-dimethylbutanamide obtained from a liquid vapor diffusion method (Experiment IDs 810055-07-A1, 810055-07-A2, 810055-07-A3, and 810048-19-B6), Type A Ref. (Experiment ID 817506-48-A), and Impurity B ref.

FIG. 23 shows DSC thermograms of three samples of crystalline Type A of N-(3,5-difluorobenzyl)-N-hydroxy-2,2-dimethylbutanamide obtained from a liquid vapor diffusion method (Experiment IDs 810055-07-A1 (top), 810055-07-A3 (middle), and 810048-19-B6 (bottom)).

FIG. 24 shows XRPD patterns of three samples of crystalline Type A of N-(3,5-difluorobenzyl)-N-hydroxy-2,2-dimethylbutanamide obtained from a grinding method (Experiment IDs 810055-08-A1, 810055-08-A2, and 810055-08-A3), Type A Ref. (Experiment ID 817506-48-A), and Impurity B ref.

FIG. 25 shows DSC thermograms of three samples of crystalline Type A of N-(3,5-difluorobenzyl)-N-hydroxy-2,2-dimethylbutanamide obtained from a grinding method (Experiment IDs 810055-08-A2 (top), 810055-08-A1 (middle), and 810055-08-A3 (bottom)).

FIG. 26 shows XRPD patterns of crystalline Type A of N-(3,5-difluorobenzyl)-N-hydroxy-2,2-dimethylbutanamide obtained from MeOH/H₂O (Experiment ID 817506-48-A2) and Type A Ref. (Experiment ID 817506-48-A).

FIG. 27 shows a DSC thermogram (bottom) and a TGA graph (top) of crystalline Type A of N-(3,5-difluorobenzyl)-N-hydroxy-2,2-dimethylbutanamide obtained from MeOH/H₂O (Experiment ID 817506-48-A2).

FIG. 28 shows a PLM image of crystalline Type A of N-(3,5-difluorobenzyl)-N-hydroxy-2,2-dimethylbutanamide obtained from MeOH/H₂O (Experiment ID 817506-48-A2).

FIG. 29 shows XRPD patterns of crystalline Type A of N-(3,5-difluorobenzyl)-N-hydroxy-2,2-dimethylbutanamide obtained from acetone/H₂O (Experiment ID 817506-48-A1), and from EtOAc/n-heptane (Experiment ID 817506-48-A4), and Type A Ref. (Experiment ID 817506-48-A).

FIG. 30 shows XRPD patterns of crystalline Type A of N-(3,5-difluorobenzyl)-N-hydroxy-2,2-dimethylbutanamide obtained from MeOH/H₂O (Experiment ID 810048-03-A1) and Type A Ref. (Experiment ID 817506-48-A), and Impurity B ref.

FIG. 31 shows DSC thermogram of crystalline Type A of N-(3,5-difluorobenzyl)-N-hydroxy-2,2-dimethylbutanamide obtained MeOH/H₂O (Experiment ID 810048-03-A1).

FIG. 32 shows XRPD patterns of crystalline Type A of N-(3,5-difluorobenzyl)-N-hydroxy-2,2-dimethylbutanamide before solubility test (Experiment ID 817506-48-A2) and after solubility test (Experiment ID 817506-50-A), and Type A Ref. (Experiment ID 817506-48-A)

FIG. 33 shows HPLC chromatograms of Type A of N-(3,5-difluorobenzyl)-N-hydroxy-2,2-dimethylbutanamide (817506-48-A2) for stability evaluation (1): Blank; (2): 80° C./1 day; (3): 25° C./60% RH/1 week; (4): 40° C./75% RH/1 week.

FIG. 34 shows XRPD patterns of crystalline Type A of N-(3,5-difluorobenzyl)-N-hydroxy-2,2-dimethylbutanamide after stability tests, Experiment IDs 810048-01-A1 (80° C./1 day), 810048-01-A2 (25° C./60% RH/1 week), and 810048-01-A3 (40° C./75% RH/1 week), and Type A Ref. (Experiment ID 817506-48-A)

FIG. 35 shows a DSC thermogram of crystalline Type A of N-(3,5-difluorobenzyl)-N-hydroxy-2,2-dimethylbutanamide after storage at 80° C. (Experiment ID 817506-48-A2).

FIG. 36 shows a DSC thermogram of crystalline Type A of N-(3,5-difluorobenzyl)-N-hydroxy-2,2-dimethylbutanamide after storage under 25° C./60% RH (Experiment ID 817506-48-A2).

FIG. 37 shows a DSC thermogram of crystalline Type A of N-(3,5-difluorobenzyl)-N-hydroxy-2,2-dimethylbutanamide after storage under 40° C./75% RH (Experiment ID 817506-48-A2).

FIG. 38 shows HPLC chromatograms of Type A (817506-48-A2) after stability tests under white light condition (1): Blank; (2): White light 1,200,000 Lux-hrs; (3): White light control.

FIG. 39 shows HPLC chromatograms of Type A (817506-48-A2) after stability tests under UV condition (1): Blank; (2): UV 200 W-hrs/m 2; (3): UV_control.

FIG. 40 shows XRPD patterns of crystalline Type A of N-(3,5-difluorobenzyl)-N-hydroxy-2,2-dimethylbutanamide after stability tests under white light conditions, Experiment IDs 817506-48-A2 (white light), 817506-48-A2 (white light control), Type A Ref. (Experiment ID 817506-48-A), and Impurity B ref.

FIG. 41 shows XRPD patterns of crystalline Type A of N-(3,5-difluorobenzyl)-N-hydroxy-2,2-dimethylbutanamide after stability tests under UV conditions, Experiment IDs 817506-48-A2 (UV), 817506-48-A2 (UV_control), Type A Ref. (Experiment ID 817506-48-A), and Impurity B ref.

FIG. 42 shows DSC thermograms of crystalline Type A of N-(3,5-difluorobenzyl)-N-hydroxy-2,2-dimethylbutanamide after a stability test under white light condition or white light control.

FIG. 43 shows DSC thermograms of crystalline Type A of N-(3,5-difluorobenzyl)-N-hydroxy-2,2-dimethylbutanamide under UV condition or UV control.

FIG. 44 shows DVS graphs of Type A (817506-48-A2).

FIG. 45 shows XRPD patterns of crystalline Type A of N-(3,5-difluorobenzyl)-N-hydroxy-2,2-dimethylbutanamide before and after a DVS test with Type A Ref.

FIG. 46 shows a DSC thermogram of crystalline Type A of N-(3,5-difluorobenzyl)-N-hydroxy-2,2-dimethylbutanamide after a DVS test.

FIG. 47 shows XRPD patterns of crystalline Type C of N-(3,5-difluorobenzyl)-N-hydroxy-2,2-dimethylbutanamide with a Type A Ref.

FIG. 48 shows XRPD patterns of crystalline Type C of N-(3,5-difluorobenzyl)-N-hydroxy-2,2-dimethylbutanamide before drying and after drying with a Type A Ref.

FIG. 49 shows XRPD patterns of three samples (SN-045-17, YF2022-2-API micronized, and YF2022-3-API micronized) of crystalline Type A of N-(3,5-difluorobenzyl)-N-hydroxy-2,2-dimethylbutanamide. Particle sizes (D90) of the “YF2022-2-API micronized” and “YF2022-3-API micronized” samples were estimated to be about 30-40 μm.

FIG. 50 shows XRPD patterns of one sample (810048-18-B) of crystalline Type A of N-(3,5-difluorobenzyl)-N-hydroxy-2,2-dimethylbutanamide with a Type A Ref.

FIG. 51 shows a DSC thermogram (bottom) and a TGA graph (top) of one sample (810048-18-B) of crystalline Type A of N-(3,5-difluorobenzyl)-N-hydroxy-2,2-dimethylbutanamide.

FIG. 52 shows XRPD patterns of one sample (810055-01-A) of crystalline Type A of N-(3,5-difluorobenzyl)-N-hydroxy-2,2-dimethylbutanamide with a Type A Ref.

FIG. 53 shows a DSC thermogram (bottom) and a TGA graph (top) of one sample (810055-01-A) of crystalline Type A of N-(3,5-difluorobenzyl)-N-hydroxy-2,2-dimethylbutanamide.

FIG. 54 shows proton NMR of one sample (810055-01-A) of crystalline Type A of N-(3,5-difluorobenzyl)-N-hydroxy-2,2-dimethylbutanamide.

FIG. 55 shows XRPD patterns of four samples of crystalline Type A of N-(3,5-difluorobenzyl)-N-hydroxy-2,2-dimethylbutanamide and Impurity B with a Type A Ref and Impurity B Ref.

FIG. 56 shows DSC thermograms of four samples of crystalline Type A of N-(3,5-difluorobenzyl)-N-hydroxy-2,2-dimethylbutanamide with Impurity B.

### DETAILED DESCRIPTION OF THE DISCLOSURE

All publications, including but not limited to patents and patent applications, cited in this specification are herein incorporated by reference as though fully set forth. If certain content of a reference cited herein contradicts or is inconsistent with the present disclosure, the present disclosure controls.

Any one embodiment of the disclosure described herein, including those described only in one section of the specification describing a specific aspect of the disclosure, and those described only in the examples or drawings, can be combined with any other one or more embodiment(s), unless explicitly disclaimed or improper.

### I. Definitions

It is to be understood that the terminology used herein is for the purpose of describing particular embodiments only and is not intended to be limiting. Unless defined otherwise, all technical and scientific terms used herein have the same meaning as commonly understood by one of ordinary skill in the art to which the disclosure pertains.

Although any methods and materials similar or equivalent to those described herein may be used in the practice for testing of the present disclosure, exemplary materials and methods are described herein. In describing and claiming the present disclosure, the following terminology is used.

Abbreviations for certain solvents described herein are provided below.
EtOAc Ethyl acetate
THF Tetrahydrofuran
2-Me THF 2-Methyl tetrahydrofuran
DMAc Dimethylacetamide
MeOH Methanol
EtOH Ethanol
DCM Dichloromethane
IPA Isopropyl alcohol
IPAc Isopropyl acetate
MIBK Methyl isobutyl ketone
DMSO Dimethyl sulfoxide
NMP N-Methyl-2-pyrrolidone
ACN Acetonitrile
MTBE Methyl tert-butyl ether
MEK Methyl Ethyl ketone

As used herein, the singular forms “a,” “an,” and “the” include plural referents unless the content clearly dictates otherwise. Thus, for example, reference to “a compound” includes a combination of two or more compounds, and the like.

The term “about” as used herein means having a value falling within an accepted standard of error of the mean, when considered by one of ordinary skill in the art, for example ±20%, preferably ±10%, more preferably ±5%, or even more preferably ±2% of the mean.

The term “substantially the same” or “substantially shown” means that variability typical for a particular method is taken into account. For example, with reference to peak positions in the context of XRPD, DSC, TGA, DVS, and NMR, the term “substantially the same” or “substantially shown” means that typical variability in peak position and intensity are taken into account. One skilled in the art will appreciate that the peak positions will show some variability. Further, one skilled in the art will appreciate that relative peak intensities will show inter-apparatus variability as well as variability due to degree of crystallinity, particle sizes, preferred orientation, prepared sample surface, and other factors known to those skilled in the art.

The term “substantially pure” or “substantially free” with respect to a particular crystalline form of a compound means that the composition comprising the crystalline form contains less than 30%, less than 20%, less than 15%, less than 10%, less than 9%, less than 8%, less than 7%, less than 6%, less than 5%, less than 4%, less than 3%, less than 2%, less than 1%, less than 0.5%, less than 0.2%, or less than 0.1% by weight of other substances, including other crystalline or other solid state forms and/or impurities. In some embodiments, “substantially pure” or “substantially free of” refers to a substance free of other substances, including other crystalline forms, other solid state forms and/or impurities. Impurities may, for example, include by-products or left over reagents from chemical reactions, contaminants, degradation products, other crystalline forms, water, and solvents.

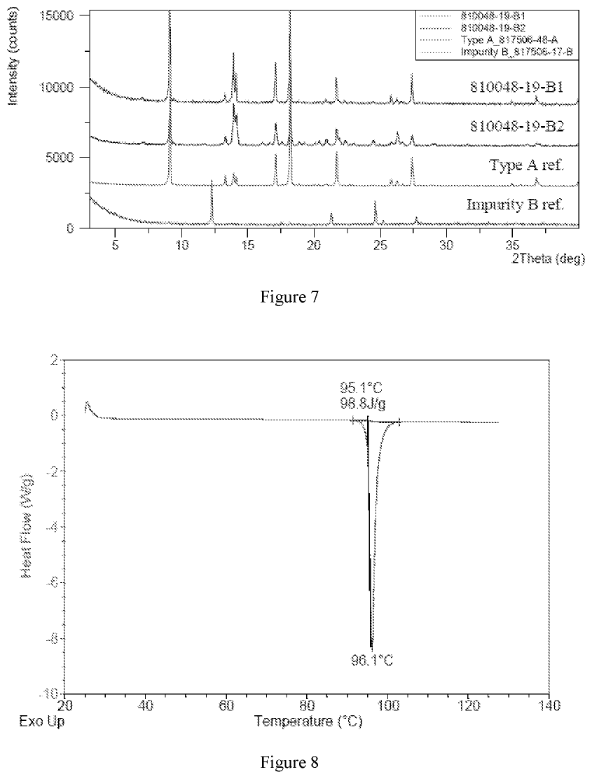

In some embodiments, a composition comprising a crystalline form of N-(3,5-difluorobenzyl)-N-hydroxy-2,2-dimethylbutanamide may also comprise an impurity, e.g., Impurity B as substantially shown in certain drawings herein, e.g., in FIG. 7. The impurity, e.g., Impurity B, may be present in a composition as disclosed herein in an amount of <5%, <4%, <3%, <2%, <1%, <0.5%, <0.2%, or <0.1% by weight. In some embodiments, Impurity B may be present in a composition as disclosed herein in about 0.1% to 0.5% by weight, such as ˜0.14% by weight.

The term “crystalline” as used herein means having a regularly repeating arrangement of molecules or external face planes. Crystalline forms may differ with respect to thermodynamic stability, physical parameters, x-ray structure, and preparation processes.

The term “micronization” as used herein refers to a process of reducing the average diameter of a solid material's particles to the micrometer range or further to the nanometer scale. A micronization process may utilize mechanical means, such as milling and grinding, or make use of the properties of supercritical fluids and manipulate the principles of solubility. A “micronized” material means that the material has gone through certain micritization process to reduce the particle sizes.

“API” as used herein means “active pharmaceutical ingredient,” such as (3,5-difluorobenzyl)-N-hydroxy-2,2-dimethylbutanamide, e.g., crystalline Type A of such compound.

The term “subject” refers to an animal including a human.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio.

The term “therapeutically effective amount” refers to the amount of a compound, e.g., a crystalline form of Formula I as disclosed herein, that produces the desired effect for which it is administered (e.g., improvement in a disease or condition, lessening the severity of a disease or condition, and/or reducing progression of a disease or condition, a disease or condition selected from an inflammatory disease, an immune disease (e.g., an autoimmune disease), an allergic disease, transplant rejection, a necrotic cell disease (e.g., a disease associated with necroptosis), a neurodegenerative disease, a CNS disease, ischemic brain injury, an ocular disease, an infectious disease, and a malignancy, including those mediated by receptor-interacting protein 1 (RIP1) signaling; a disease or condition selected from ulcerative colitis, Crohn's disease, psoriasis, rheumatoid arthritis, ALS, Alzheimer's disease, and a viral infection, including those mediated by RIP1 signaling; a disease or condition mediated by RIP1 signaling. The exact amount of a therapeutically effective amount will depend on the purpose of the treatment and will be ascertainable by one skilled in the art using known techniques (see, e.g., Lloyd (1999), The Art, Science and Technology of Pharmaceutical Compounding).

As used herein, the term “treatment” and its cognates refer to slowing or stopping disease progression. “Treatment” and its cognates as used herein include, but are not limited to the following: complete or partial remission, curing a disease or condition or a symptom thereof, lower risk of a disease or condition, a disease or condition selected from an inflammatory disease, an immune disease (e.g., an autoimmune disease), an allergic disease, transplant rejection, a necrotic cell disease, a neurodegenerative disease, a CNS disease, ischemic brain injury, an ocular disease, an infectious disease, and a malignancy, including those mediated by receptor-interacting protein 1 (RIP1) signaling; a disease or condition selected from ulcerative colitis, Crohn's disease, psoriasis, rheumatoid arthritis, ALS, Alzheimer's disease, and a viral infection, including those mediated by receptor-interacting protein 1 (RIP1) signaling; a disease or condition mediated by RIP1 signaling. Improvements in or lessening the severity of any of these symptoms can be assessed according to methods and techniques known in the art.

The term “N-(3,5-difluorobenzyl)-N-hydroxy-2,2-dimethylbutanamide,” or “Formula I,” as used herein, refers to the compound or one or more variations of the compound, e.g., tautomers, solvates (e.g., hydrate), and pharmaceutically acceptable salts of the foregoing. The compound, tautomers, solvates (e.g., hydrate), and pharmaceutically acceptable salts may also contain unnatural proportions of atomic isotopes at one or more of the atoms that constitute such compounds, such as deuterium, e.g., —CD₃, CD₂H or CDH₂ in place of methyl. For example, the compounds may be radiolabeled with radioactive isotopes, such as for example tritium (³H) or carbon-14 (¹⁴C). All isotopic variations of the compounds of the disclosure, whether radioactive or not, are intended to be encompassed within the scope of the disclosure.

### II. Crystalline Forms

One aspect of this disclosure provides solid state forms, e.g., crystalline or amorphous forms, of N-(3,5-difluorobenzyl)-N-hydroxy-2,2-dimethylbutanamide (Formula I).

In some embodiments, the present disclosure provides crystalline Type A of Formula I, such as crystalline Type A substantially free of other solid forms of Formula I.

In some embodiments, the present disclosure provides crystalline Type C of Formula I, such as crystalline Type C substantially free of other solid forms of Formula I.

In some embodiments, the present disclosure provides an amorphous form of Formula I, such as an amorphous form substantially free of other solid forms of Formula I.

In some embodiments, the present disclosure provides a composition comprising crystalline Type A of Formula I. In some embodiments, the present disclosure provides a composition comprising crystalline Type C of Formula I.

In some embodiments, the present disclosure provides a composition comprising crystalline Type A and an amorphous form of Formula I.

In some embodiments, the present disclosure provides a composition comprising crystalline Type A and crystalline Type C of Formula I.

In some embodiments, the present disclosure provides a composition comprising crystalline Type A, crystalline Type C, and amorphous form of Formula I.

Techniques for characterizing the crystalline forms of this disclosure include, but are not limited to, powder X-ray diffractometry (XRPD), differential scanning calorimetry (DSC), thermal gravimetric analysis (TGA), dynamic vapor sorption (DVS), polarized light microscopy (PLM), vibrational spectroscopy (e.g., IR and Raman spectroscopy), scanning electron microscopy, solid state NMR (Nuclear Magnetic Resonance), hot stage optical microscopy, electron crystallography, single crystal X-ray diffractometry, quantitative analysis, particle size analysis (e.g., particle size, particle size distribution, and particle shape), specific surface area analysis, surface energy analysis (e.g., inverse gas chromatography or IGC), solubility studies and dissolution studies, or a combination of these techniques.

In some embodiments, the crystalline forms are characterized by an X-ray powder diffraction pattern (XRPD). The diffractogram of XRPD is typically represented by a diagram plotting the intensity of the peaks versus the location of the peaks, i.e., diffraction angle 26 (two-theta) in degrees. The characteristic peaks of a given XRPD can be selected according to the peak locations and their relative intensity to distinguish this crystalline structure from others. Those skilled in the art recognize that the measurements of the XRPD peak locations and/or intensity for a given crystalline form of the same compound will vary as disclosed herein. The values of degree 2θ allow appropriate variations.

Differences in XRPD patterns among separate measurements of the same polymorph may arise for many reasons. Sources of variations include variations in sample preparation (e.g., sample height), instrument variations, particle size variations, calibration variations, and operator variations (including variations in determining peak locations). Preferential orientation, i.e., a lack of random orientation of crystals in the XRPD sample, can result in significant differences in relative peak heights. Particle size can also cause significant differences in relative peak heights. For example, in general, the smaller the particle sizes are, the stronger the signals of the diffraction peaks are. Calibration errors and sample height errors often result in a shift of all of the peaks of the diffractogram in the same direction and by the same amount. Small differences in sample height on a flat holder may lead to large displacements in XRPD peak positions. For a systematic study showing that sample height differences of 1 mm may lead to peak shifts as high as 1 to 28, see Chen et al., J. Pharmaceutical and Biomedical Analysis 30 (2001) 26:63.

Typically, the error margins are represented by “±”. For example, “(±0.2 degrees 2θ) at 8.7” means “at the degree 20 of about 8.7±0.2,” denoting a range from about (8.7+0.2), i.e., 8.9, to about (8.7-0.2), i.e., about 8.5. Depending on the sample preparation techniques, the calibration techniques applied to the instruments, human operational variations, and etc., those skilled in the art recognize that the appropriate error of margins for a XRPD can be ±0.5; ±0.4; ±0.3; ±0.2; ±0.1; ±0.05; or less. In some embodiments of this disclosure, the XRPD margin of error is ±0.2. In some embodiments of this disclosure, the XRPD margin of error is ±0.5.

In many instances, peak shifts among diffraction patterns resulting from systematic error can be eliminated by compensating for the shift (e.g., applying a correction factor to all peak position values) or by recalibrating the diffractometer. Generally, the same techniques can be used to compensate for differences among diffractometers so that XRPD peak positions obtained from two different instruments can be brought into agreement. Furthermore, when these techniques are applied to XRPD measurements from the same or different diffractometers, the peak positions for a particular polymorph will usually agree to within about ±0.2.

### Type A of Formula I

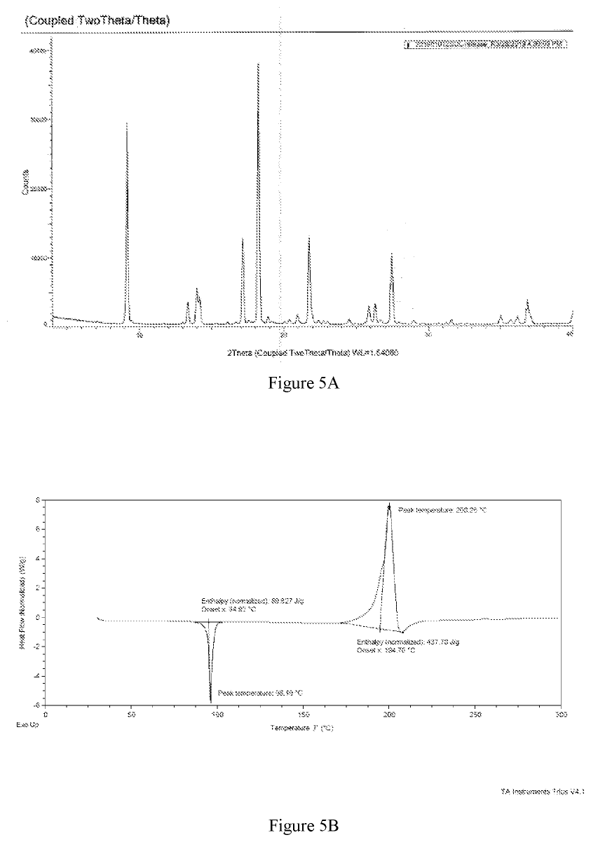

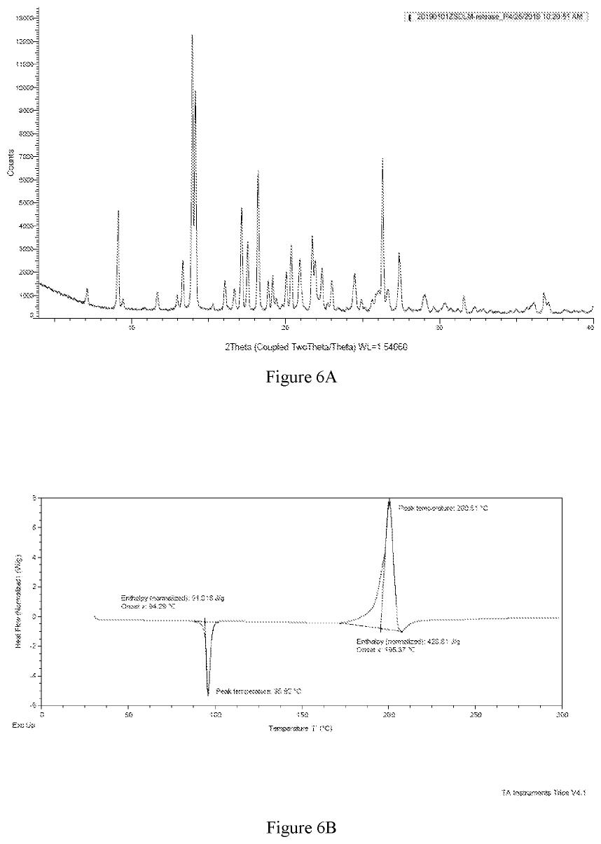

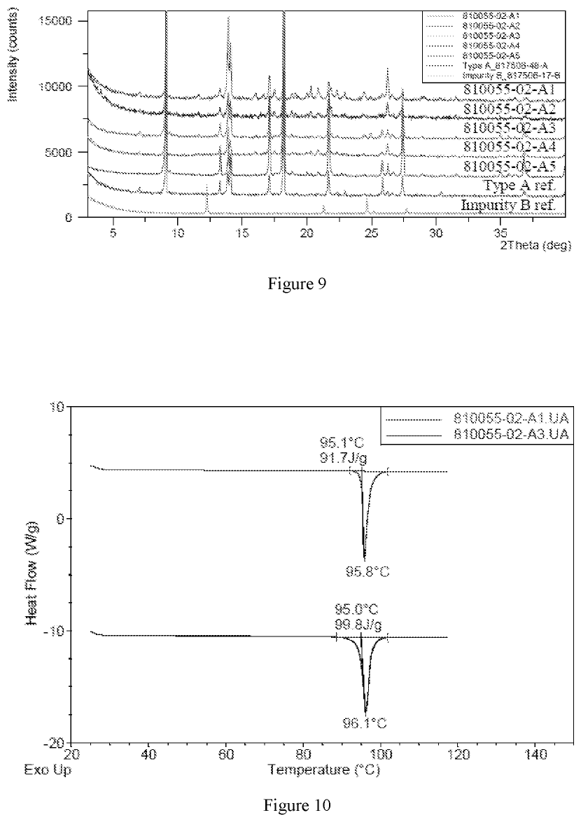

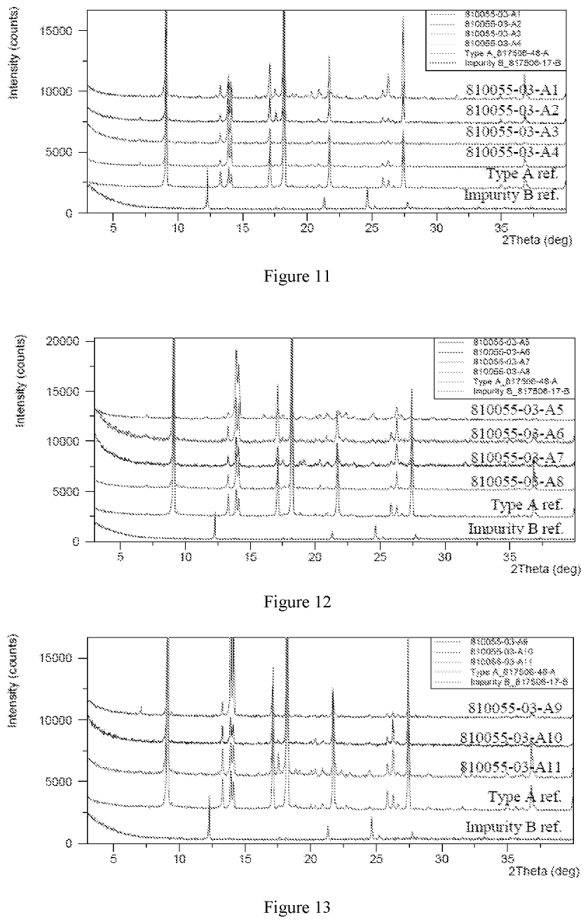

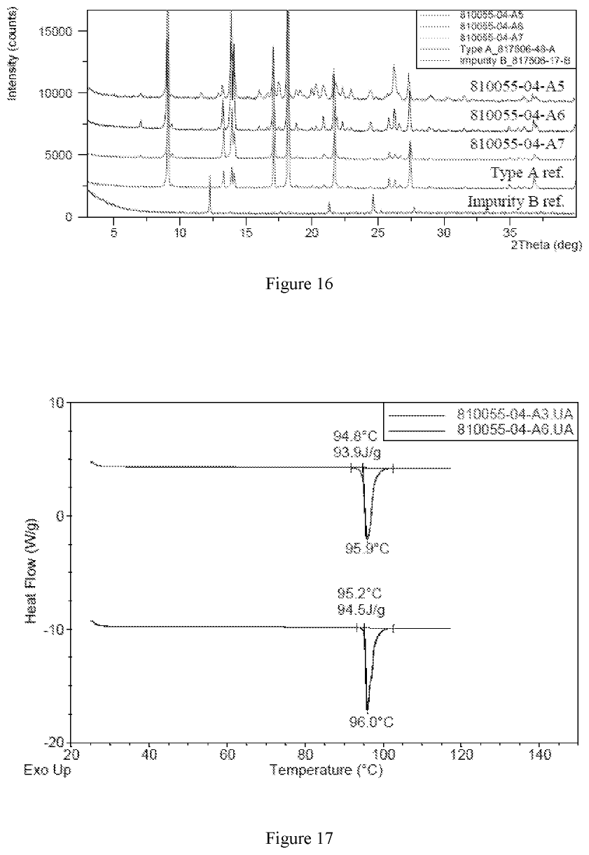

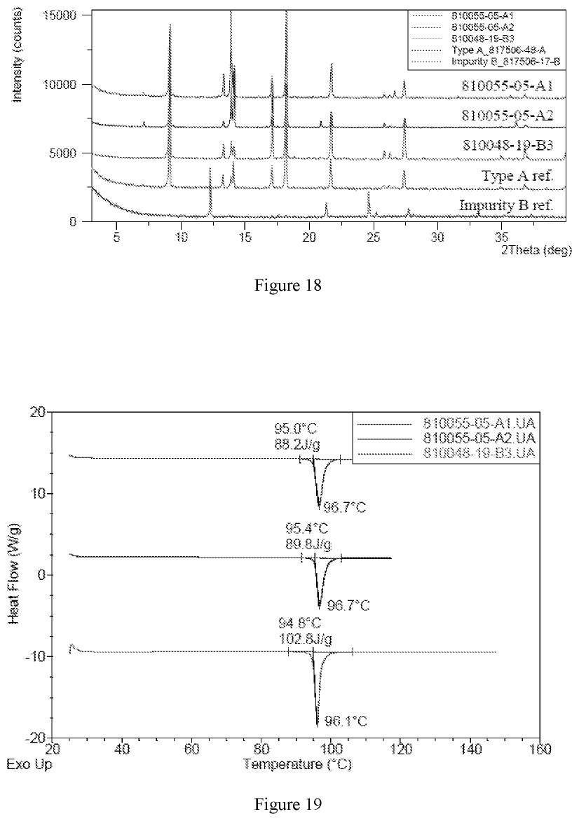

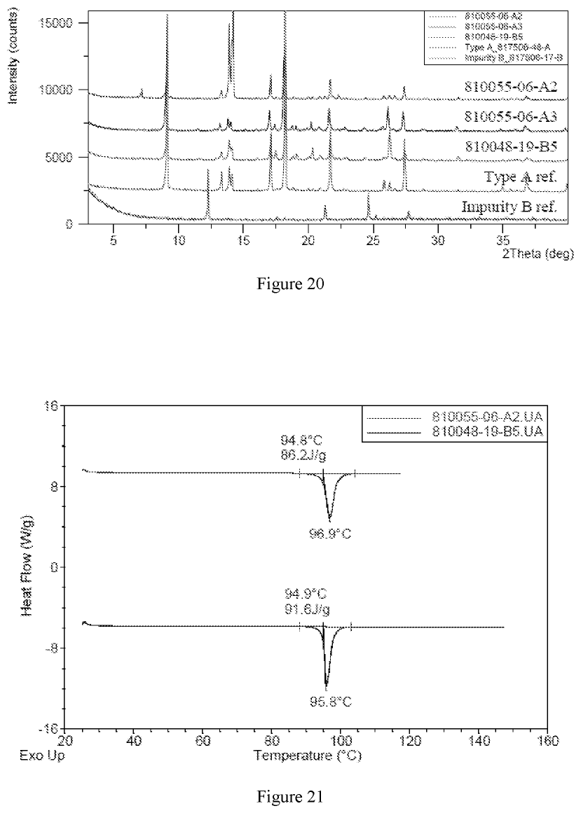

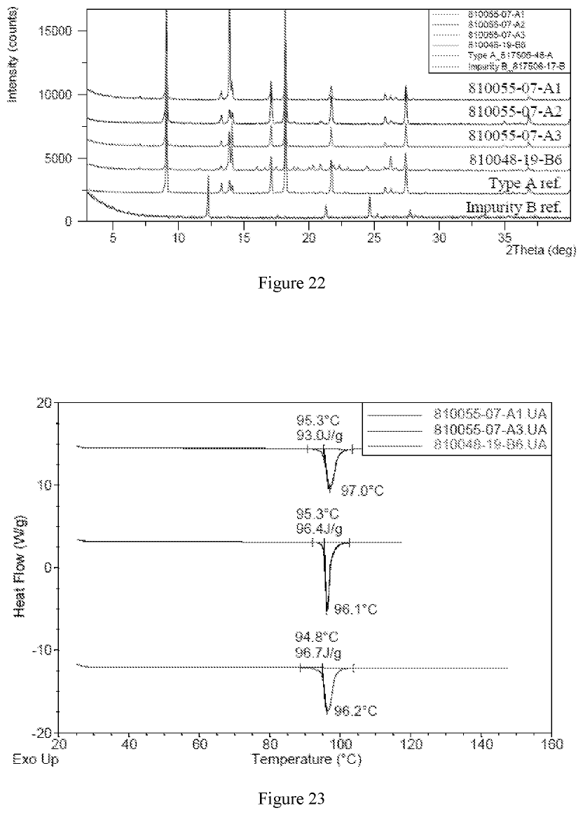

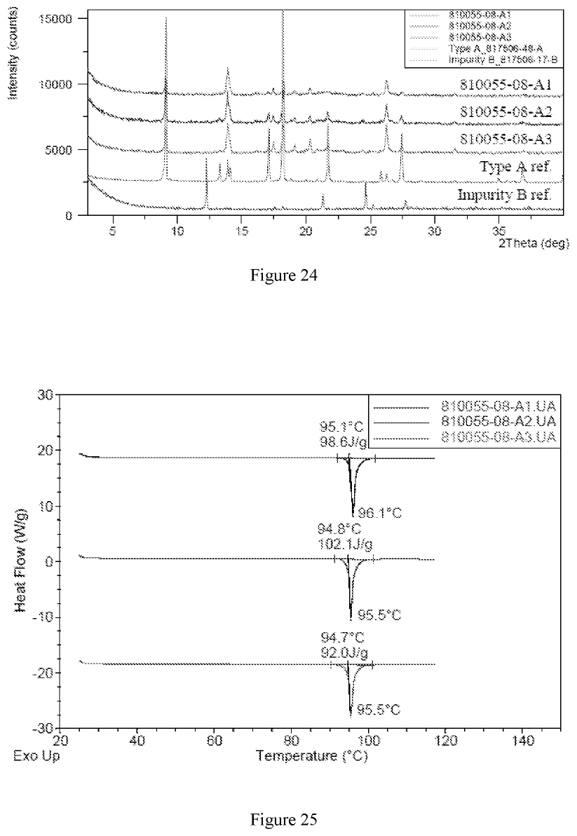

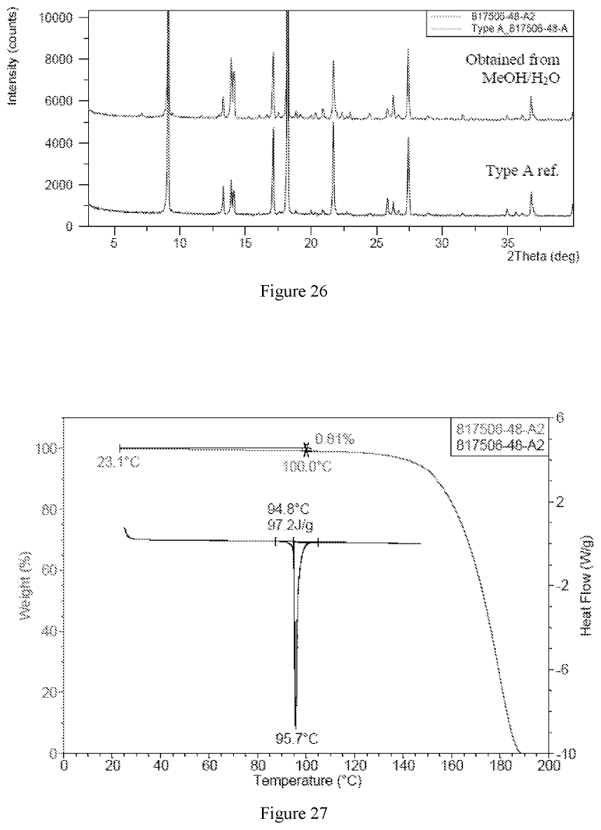

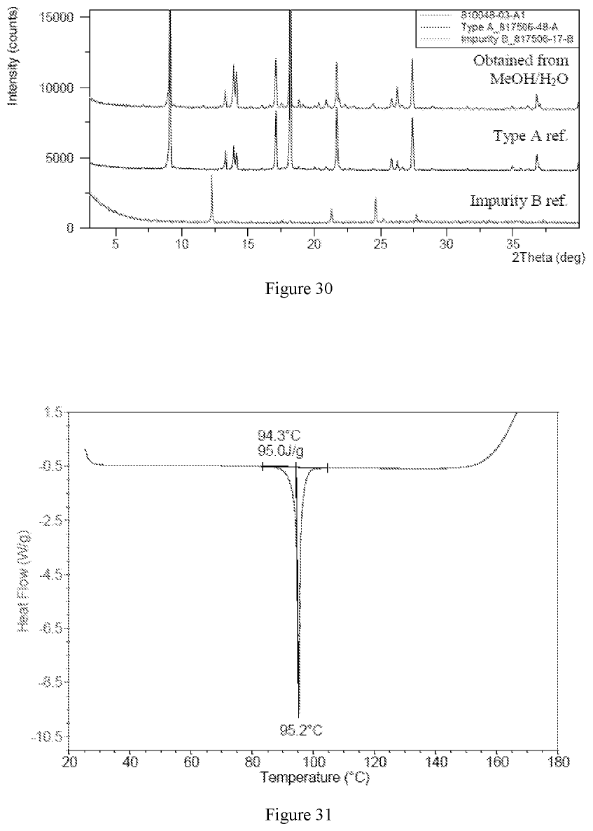

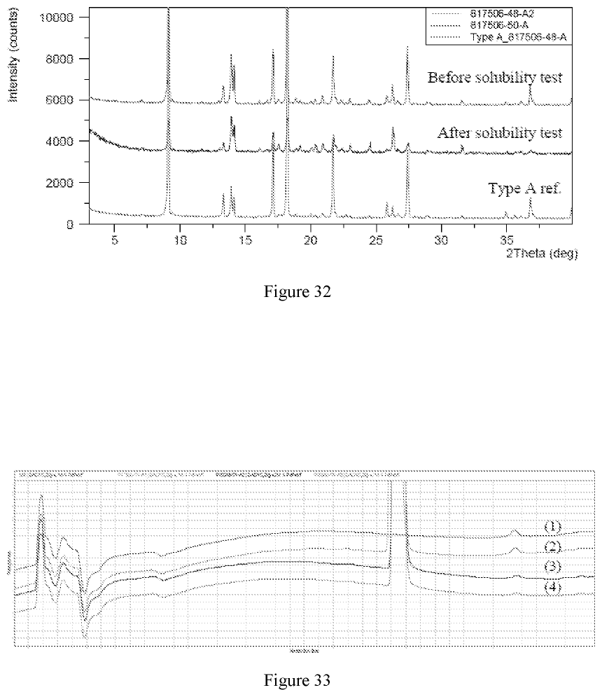

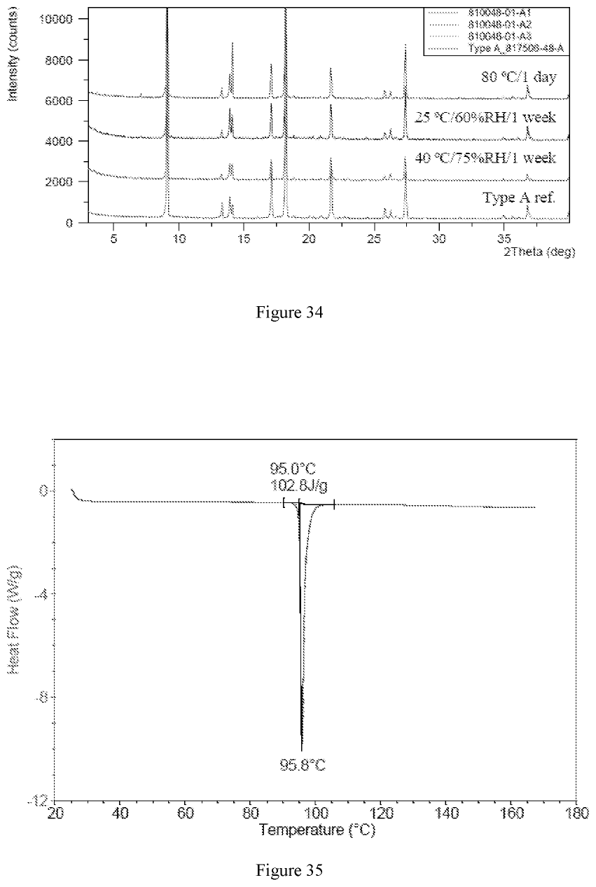

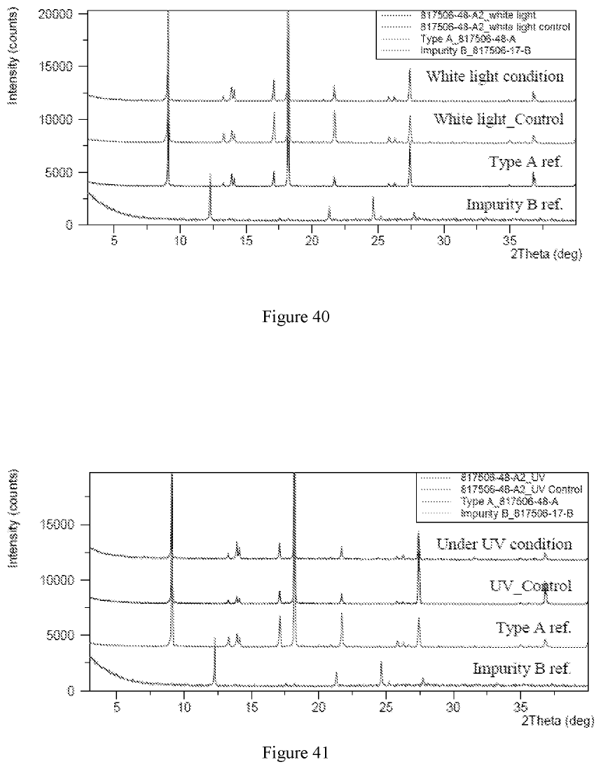

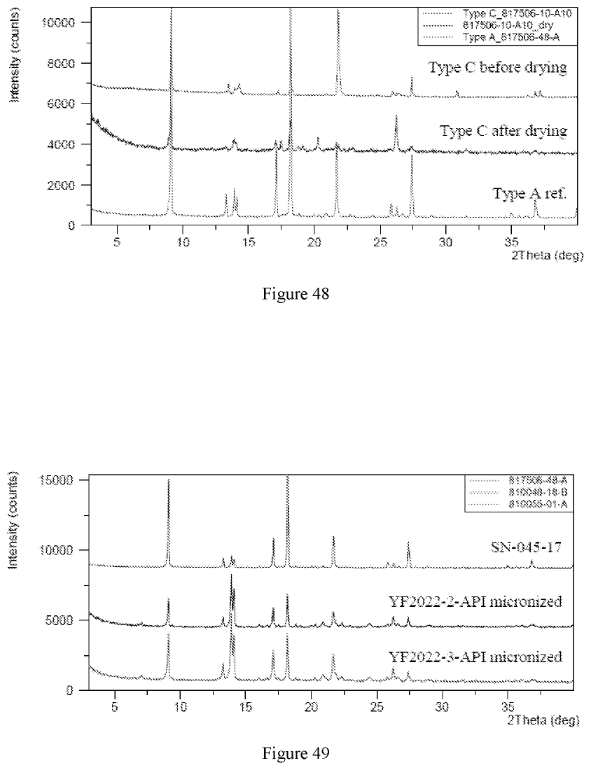

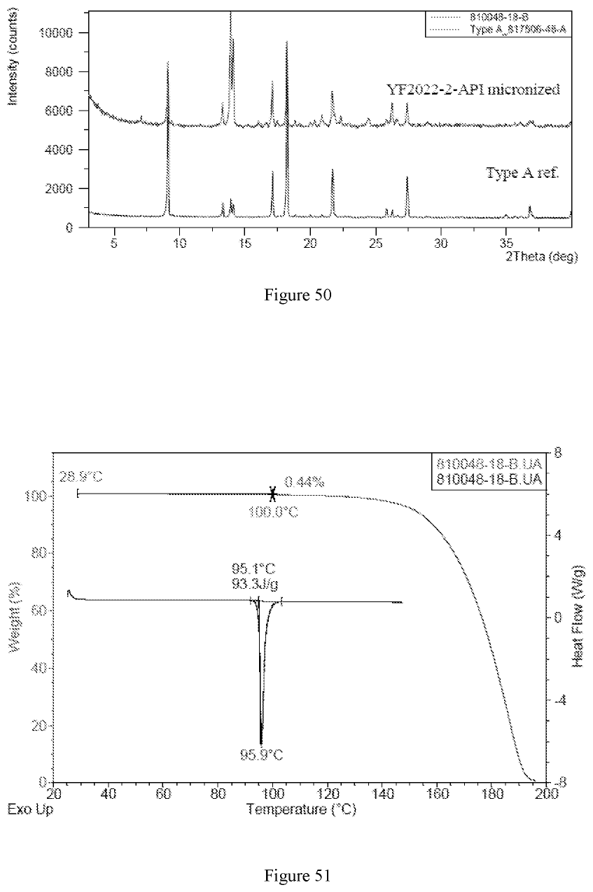

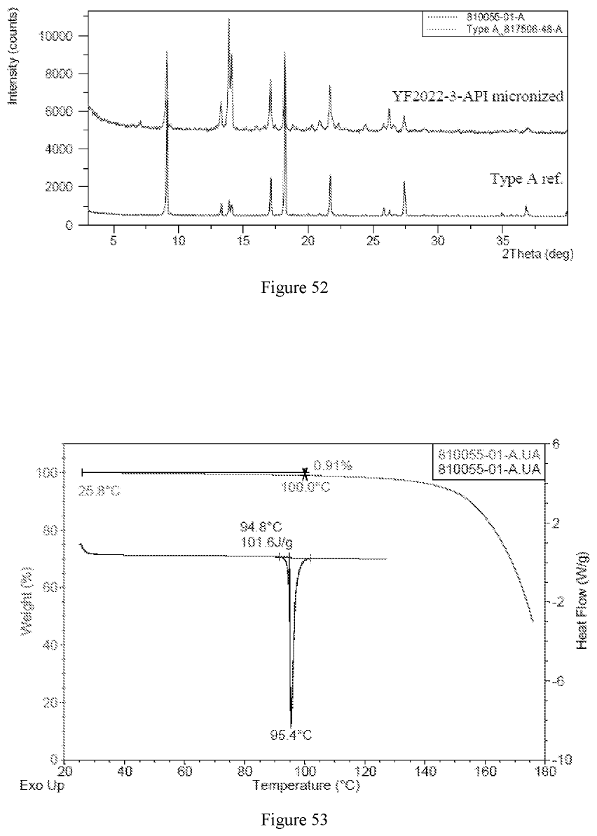

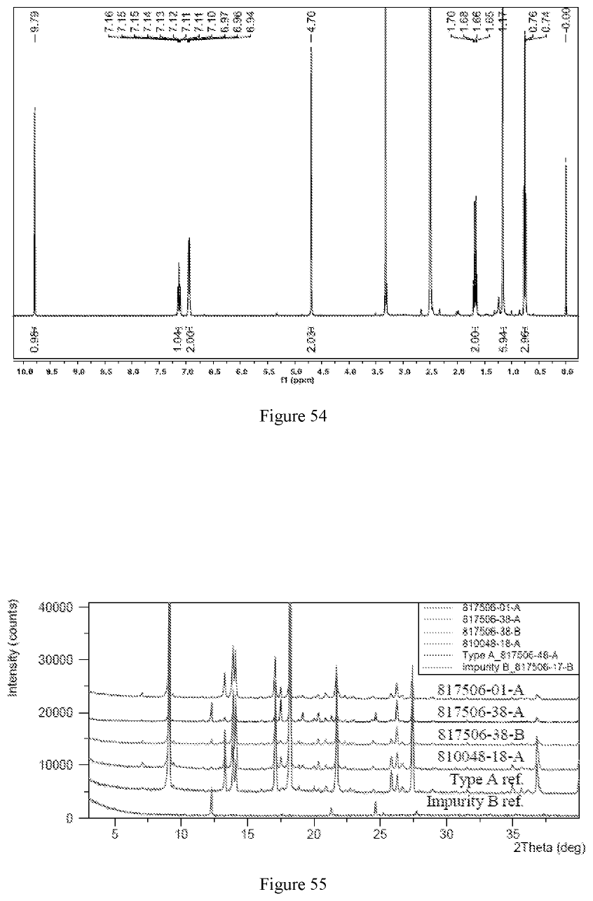

In some embodiments, crystalline Type A of Formula I has an XRPD pattern substantially the same as one of the XRPD patterns indicated for crystalline Type A of N-(3,5-difluorobenzyl)-N-hydroxy-2,2-dimethylbutanamide shown in FIG. 1, FIG. 5A, FIG. 6A, FIG. 7, FIG. 9, FIG. 11, FIG. 12, FIG. 13, FIG. 15, FIG. 16, FIG. 18, FIG. 20, FIG. 22, FIG. 24, FIG. 26, FIG. 29, FIG. 30, FIG. 32, FIG. 34, FIG. 40, FIG. 41, FIG. 45, FIG. 47, FIG. 48, FIG. 49, FIG. 50, FIG. 52, and FIG. 55.

In some embodiments, crystalline Type A of Formula I has an XRPD pattern characterized by at least two, at least three, at least four, at least five, at least six, at least seven, at least eight, or at least nine of the degree 2θ-peaks with the greatest intensity as substantially shown in one of the XRPD patterns indicated for crystalline Type A of N-(3,5-difluorobenzyl)-N-hydroxy-2,2-dimethylbutanamide shown in FIG. 1, FIG. 5A, FIG. 6A, FIG. 7, FIG. 9, FIG. 11, FIG. 12, FIG. 13, FIG. 15, FIG. 16, FIG. 18, FIG. 20, FIG. 22, FIG. 24, FIG. 26, FIG. 29, FIG. 30, FIG. 32, FIG. 34, FIG. 40, FIG. 41, FIG. 45, FIG. 47, FIG. 48, FIG. 49, FIG. 50, FIG. 52, and FIG. 55.

In some embodiments, crystalline Type A of Formula I has an XRPD pattern characterized by at least two, at least three, at least four, at least five, at least six, at least seven, at least eight, at least nine, at least ten, or at least eleven of the degree 2θ-peaks with the greatest intensity as substantially shown in the XRPD pattern in FIG. 5A.

In some embodiments, crystalline Type A of Formula I has an XRPD pattern characterized by at least two, at least three, at least four, at least five, at least six, at least seven, at least eight, at least nine, at least ten, or at least eleven of the degree 2θ-peaks with the greatest intensity as substantially shown in the XRPD pattern in FIG. 6A.

In some embodiments, crystalline Type A of Formula I has an XRPD pattern comprising degree 2θ-peaks (±0.2 degrees 2θ) at 13.96 and one of the degree 20-peaks (±0.2 degrees 2θ) at 9.15, 14.15, 17.15, 18.22, and 26.31. In some embodiments, crystalline Type A of Formula I has an XRPD pattern comprising degree 2θ-peaks (±0.2 degrees 2θ) at 13.96 and two of the degree 2θ-peaks (±0.2 degrees 2θ) at 9.15, 14.15, 17.15, 18.22, and 26.31. In some embodiments, crystalline Type A of Formula I has an XRPD pattern comprising degree 2θ-peaks (±0.2 degrees 2θ) at 13.96 and 14.15. In some embodiments, crystalline Type A of Formula I has an XRPD pattern comprising any three degree 2θ-peaks (±0.2 degrees 2θ) selected from the group consisting of 9.15, 13.96, 14.15, 17.15, 18.22, and 26.31.

In some embodiments, crystalline Type A of Formula I has an XRPD pattern comprising degree 2θ-peaks (±0.2 degrees 2θ) at 18.20 and one of the degree 20-peaks (±0.2 degrees 2θ) at 9.10, 17.10, 21.7, and 27.40. In some embodiments, crystalline Type A of Formula I has an XRPD pattern comprising degree 2θ-peaks (±0.2 degrees 2θ) at 18.20 and two of the degree 2θ-peaks (±0.2 degrees 2θ) at 9.10, 17.10, 21.7, and 27.40. In some embodiments, crystalline Type A of Formula I has an XRPD pattern comprising degree 2θ-peaks (±0.2 degrees 2θ) at 9.10 and 18.20. In some embodiments, crystalline Type A of Formula I has an XRPD pattern comprising any three degree 2θ-peaks (±0.2 degrees 2θ) selected from the group consisting of 9.10, 17.10, 18.20, 21.7, and 27.40.

In some embodiments, crystalline Type A of Formula I has an XRPD pattern comprising degree 20 (2 theta)-peaks (±0.2 degrees 2θ) at 9.15, 17.15 and 18.22.

In some embodiments, crystalline Type A of Formula I has an XRPD pattern comprising degree 20 (2 theta)-peaks (±0.2 degrees 2θ) at 9.15, 17.15, 18.22, 21.8, and 27.40. In some embodiments, crystalline Type A of Formula I has an XRPD pattern comprising degree 20 (2 theta)-peaks (±0.2 degrees 2θ) at 9.15, 13.34, 17.15, 18.22, 21.80, and 27.40.

In some embodiments, crystalline Type A of Formula I has an XRPD pattern comprising degree 2θ-peaks (±0.2 degrees 2θ) at 13.96, 14.15, and 26.31. In some embodiments, crystalline Type A of Formula I has an XRPD pattern comprising degree 2θ-peaks (±0.2 degrees 2θ) at 13.96, 14.15, 18.22, and 26.31. In some embodiments, crystalline Type A of Formula I has an XRPD pattern comprising degree 2θ-peaks (±0.2 degrees 2θ) at 13.96, 14.15, 17.15, 18.22, and 26.31. In some embodiments, crystalline Type A of Formula I has an XRPD pattern comprising degree 2θ-peaks (±0.2 degrees 2θ) at 9.15, 13.96, 14.15, 17.15, 18.22, and 26.31. In some embodiments, crystalline Type A of Formula I has an XRPD pattern comprising degree 2θ-peaks (±0.2 degrees 2θ) at 9.15, 13.96, 14.15, 17.15, 18.22, 20.38, 26.31, and 27.40. In some embodiments, crystalline Type A of Formula I has an XRPD pattern comprising degree 2θ-peaks (±0.2 degrees 2θ) at 9.15, 13.34, 13.96, 14.15, 17.15, 18.22, 20.38, 20.93, 21.80, 21.93, 26.31, and 27.40.

In some embodiments, crystalline Type A of Formula I has an XRPD pattern comprising degree 2θ-peaks (±0.2 degrees 2θ) at 9.10, 17.10, and 18.20. In some embodiments, crystalline Type A of Formula I has an XRPD pattern comprising degree 2θ-peaks (±0.2 degrees 2θ) at 9.10, 17.10, 18.20, and 21.7. In some embodiments, crystalline Type A of Formula has an XRPD pattern comprising degree 2θ-peaks (±0.2 degrees 2θ) at 9.10, 17.10, 18.20, 21.7, and 27.40. In some embodiments, crystalline Formula I Form I has an XRPD pattern comprising degree 2θ-peaks (±0.2 degrees 2θ) at 9.10, 13.9, 17.10, 18.20, 21.7, and 27.40.

In one embodiment, crystalline Type A of Formula I comprises the XRPD peaks substantially shown below in Table 1. The XRPD peaks as shown in Table 1 was obtained from a crystalline sample having a particle size (D90) of about 15 μm.

TABLE 1

| Peak Position (degree 2θ) | Relative Intensity (%) |
|---|---|
| 7.12 | 5.5 |
| 9.15 | 35.8 |
| 9.45 | 3.6 |
| 10.82 | 0.6 |
| 11.69 | 6.3 |
| 12.97 | 5.5 |
| 13.34 | 18.0 |
| 13.96 | 100.0 |
| 14.15 | 80.2 |
| 15.28 | 2.4 |
| 16.07 | 10.6 |
| 16.68 | 7.6 |
| 17.15 | 37.0 |
| 17.55 | 24.6 |
| 18.22 | 50.7 |
| 18.89 | 10.5 |
| 19.17 | 12.5 |
| 19.41 | 3.9 |
| 20.05 | 13.4 |
| 20.38 | 23.6 |
| 20.93 | 18.6 |
| 21.80 | 16.2 |
| 21.93 | 18.2 |
| 22.37 | 15.2 |
| 23.01 | 10.8 |
| 23.40 | 0.8 |
| 24.07 | 0.5 |
| 24.50 | 13.2 |
| 24.94 | 3.8 |
| 25.63 | 3.9 |
| 26.03 | 6.9 |
| 26.31 | 55.1 |
| 26.65 | 7.5 |
| 27.40 | 20.1 |
| 28.01 | 1.2 |
| 29.05 | 6.1 |
| 29.63 | 2.1 |
| 30.35 | 3.1 |
| 30.73 | 1.2 |
| 31.20 | 1.1 |
| 31.58 | 5.9 |
| 32.29 | 2.1 |
| 32.85 | 1.4 |
| 33.30 | 0.9 |
| 33.76 | 0.4 |
| 34.62 | 0.9 |
| 34.97 | 1.3 |
| 35.65 | 1.9 |
| 36.12 | 3.7 |
| 36.86 | 5.8 |
| 37.07 | 3.8 |
| 38.14 | 1.2 |
| 38.80 | 0.4 |
| 39.24 | 1.0 |

In one embodiment, crystalline Type A of Formula I comprises the XRPD peaks substantially shown below in Table 2.

TABLE 2

| Peak Position (degree 2θ) | Relative Intensity (%) |
|---|---|
| 9.10 | 77.2 |
| 9.38 | 1.2 |
| 13.29 | 8.4 |
| 13.90 | 10.8 |
| 14.10 | 7.0 |
| 16.03 | 0.6 |
| 17.10 | 25.7 |
| 17.56 | 0.8 |
| 18.20 | 100.0 |
| 18.84 | 1.3 |
| 20.00 | 1.3 |
| 20.32 | 0.8 |
| 20.88 | 1.5 |
| 21.70 | 27.2 |
| 22.70 | 0.99 |
| 22.98 | 0.5 |
| 24.44 | 0.7 |
| 25.82 | 5.2 |
| 26.26 | 4.2 |
| 26.65 | 1.5 |
| 27.40 | 23.6 |
| 28.86 | 0.7 |
| 31.55 | 0.8 |
| 34.92 | 2.1 |
| 35.60 | 1.1 |
| 36.78 | 7.1 |

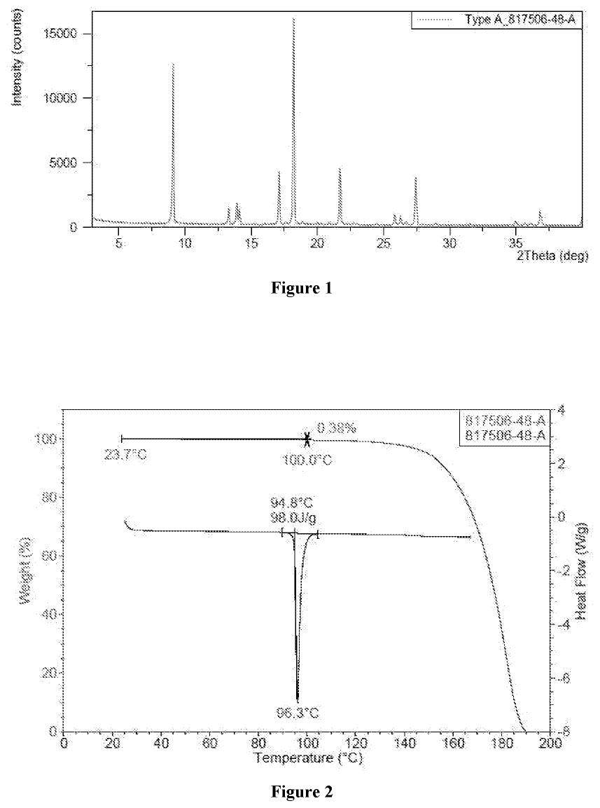

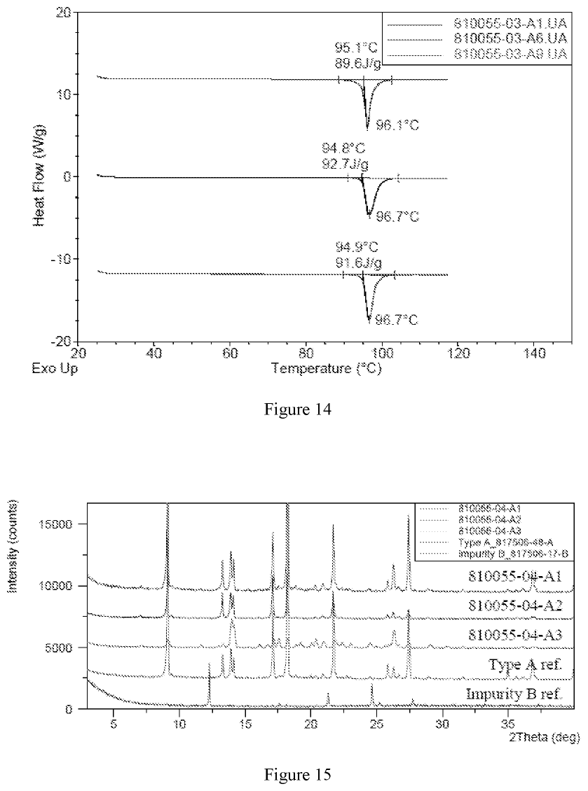

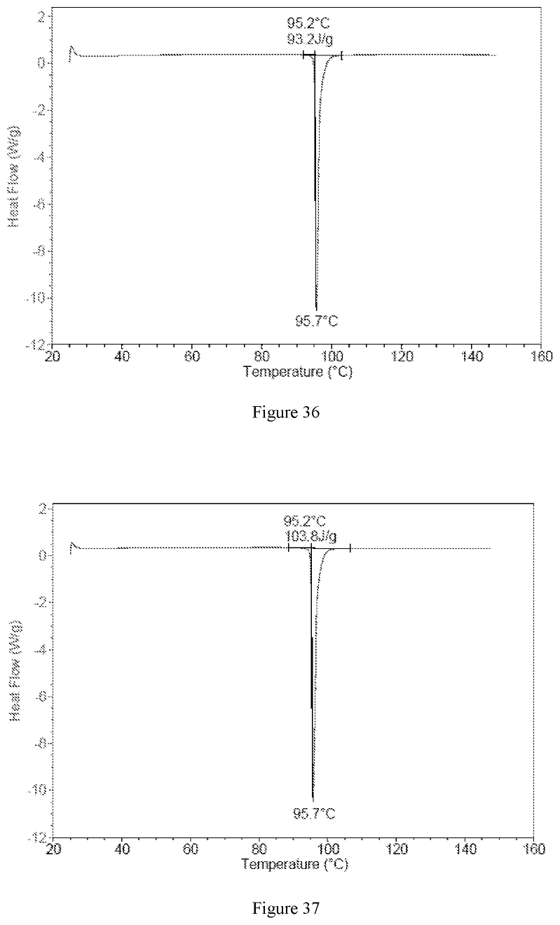

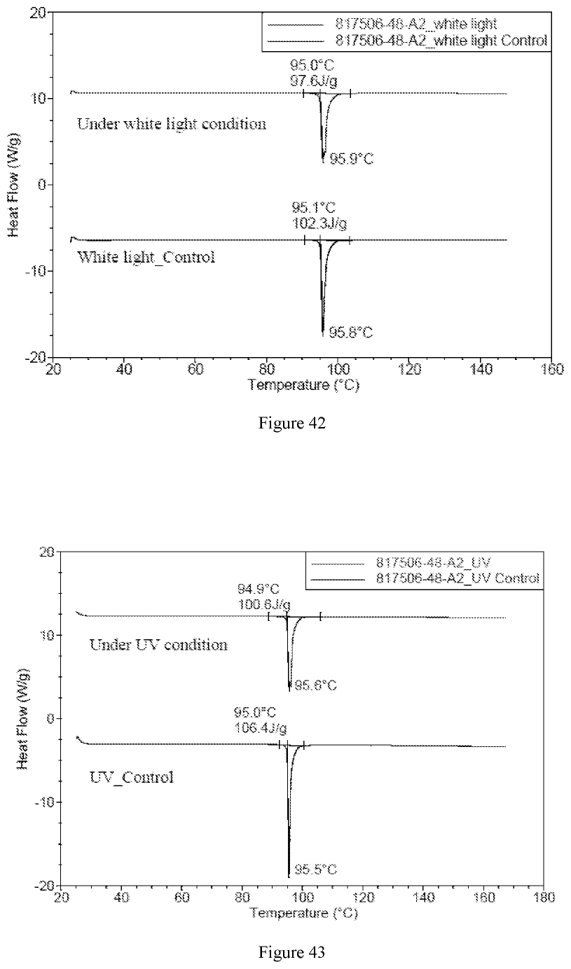

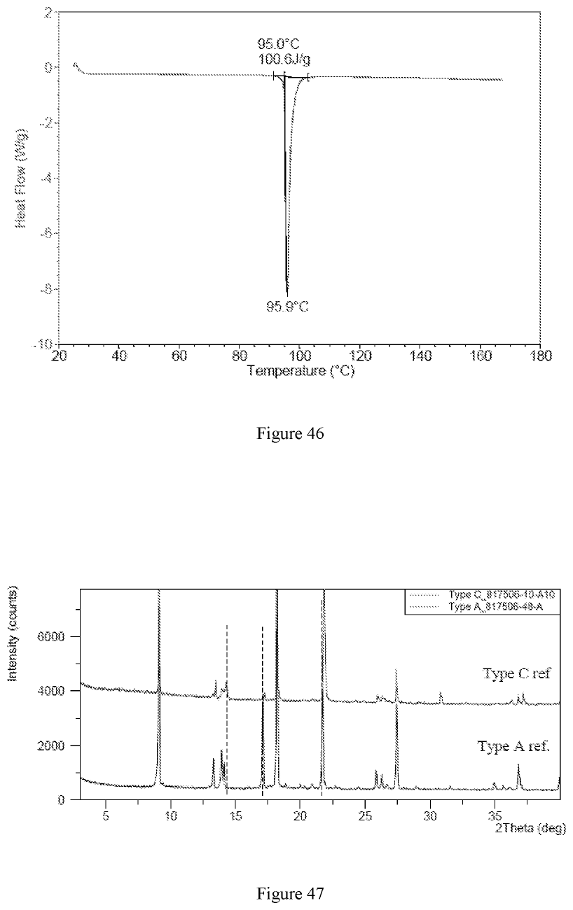

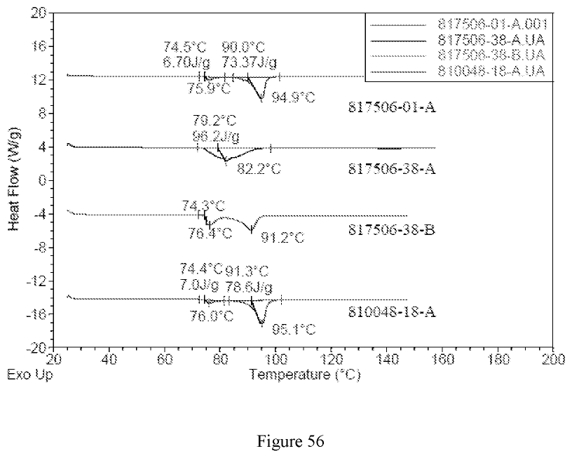

In some embodiments, crystalline Type A of Formula I may exhibit a DSC thermogram, before decomposition of the compound, substantially the same as one of the DSC thermograms shown in FIG. 2, FIG. 5B (before decomposition of the compound), FIG. 6B (before decomposition of the compound), FIG. 8, FIG. 10, FIG. 14, FIG. 17, FIG. 19, FIG. 21, FIG. 23, FIG. 25, FIG. 27, FIG. 31, FIG. 35, FIG. 36, FIG. 37, FIG. 42, FIG. 43, FIG. 46, FIG. 51, FIG. 53, and FIG. 56. In certain embodiment, the crystalline Type A has an onset melting temperature of from about 94° C. to about 96° C. In certain embodiment, crystalline Type A has an onset melting temperature of from about 94.5° C. to about 95.5° C. In certain embodiment, the crystalline Type A has an onset melting temperature of about 95° C. In certain embodiment, the crystalline Type A has an onset melting temperature of about 94.8° C. In certain embodiment, the crystalline Type A has an onset melting temperature of about 95.1° C.

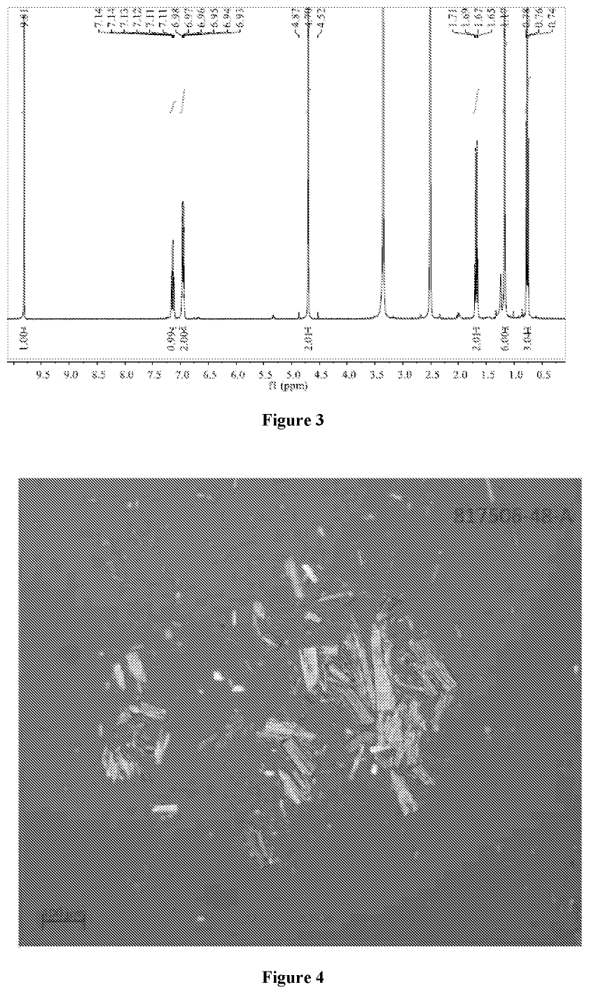

In some embodiments, crystalline Type A of Formula I may exhibit a TGA graph substantially the same as one of the TGA graphs shown in FIG. 3, FIG. 27, FIG. 51, and FIG. 53.

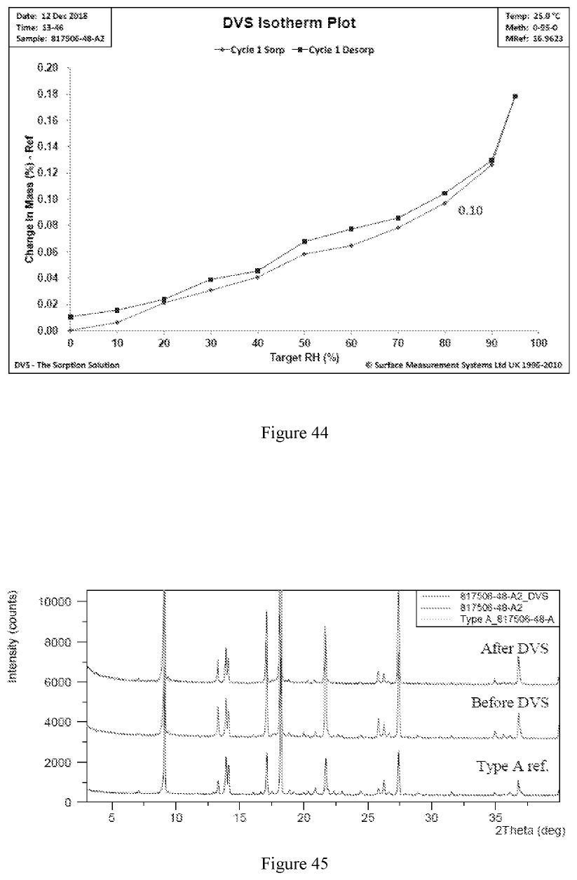

In some embodiments, crystalline Type A of Formula I may exhibit a DVS graph substantially the same as one of the DVS graphs shown in FIG. 44.

In some embodiments of crystalline Type A of Formula I, at least one, at least two, at least three, or all of the following (a)-(d) apply to the crystalline Type A of Formula I: (a) an XRPD pattern substantially the same as shown as one of the XRPD patterns indicated for crystalline Type A of N-(3,5-difluorobenzyl)-N-hydroxy-2,2-dimethylbutanamide shown in FIG. 1, FIG. 5A, FIG. 6A, FIG. 7, FIG. 9, FIG. 11, FIG. 12, FIG. 13, FIG. 15, FIG. 16, FIG. 18, FIG. 20, FIG. 22, FIG. 24, FIG. 26, FIG. 29, FIG. 30, FIG. 32, FIG. 34, FIG. 40, FIG. 41, FIG. 45, FIG. 47, FIG. 48, FIG. 49, FIG. 50, FIG. 52, and FIG. 55; (b) a DSC thermogram, before decomposition of the compound, substantially the same as one of the DSC thermograms shown in FIG. 2, FIG. 5B (before decomposition of the compound), FIG. 6B (before decomposition of the compound), FIG. 8, FIG. 10, FIG. 14, FIG. 17, FIG. 19, FIG. 21, FIG. 23, FIG. 25, FIG. 27, FIG. 31, FIG. 35, FIG. 36, FIG. 37, FIG. 42, FIG. 43, FIG. 46, FIG. 51, FIG. 53, and FIG. 56; (c) a TGA graph substantially the same as one of the TGA graphs shown in FIG. 3, FIG. 27, FIG. 51, and FIG. 53; (d) a DVS graph substantially the same as one of the DVS graphs shown in FIG. 44.

In some embodiments, crystalline Type A of Formula I has the following properties: (a) an XRPD pattern substantially the same as shown in in FIG. 1; (b) a DSC thermogram substantially the same as shown in FIG. 2; and (c) a TGA graph substantially the same as shown in FIG. 2.

In some embodiments, crystalline Type A of Formula I has the following properties: (a) an XRPD pattern substantially the same as shown in in FIG. 6A; and (b) a DSC thermogram substantially the same as shown in FIG. 6B.

In some embodiments, crystalline Type A of Formula I is anhydrous.

In some embodiments, crystalline Type A of Formula I is non-hygroscopic.

In certain embodiment, dynamic vapor sorption (DVS) data of crystalline Type A of Formula I shows a water uptake of about 0.10% at 80% RH/25° C.

In some embodiments, an equilibrium solubility of crystalline Type A of Formula I in H2O at room temperature is about 0.15 mg/ml.

Crystalline Type A of Formula I is substantially stable. In certain embodiment, no crystalline type change is detected after suspension of crystalline Type A of Formula I in H2O for 24 hrs. In certain embodiment, solid-state stability of Type A is evaluated at 80° C. for one day, 25° C./60% RH for one week, and 40° C./75% RH for one week. No crystalline form change or HPLC purity decrease is detected under these conditions. In certain embodiment, photo stability of Type A is evaluated under both white light (1,200,000 Lux•hrs) and UV (200 W•hrs/m²) conditions. No crystalline form change or HPLC purity decrease is observed under either condition.

As further illustrated in the Examples, crystalline Type A of Formula I can be made via methods such as anti-solvent addition, solid vapor diffusion, solution vapor diffusion, slurry, slow evaporation, slow cooling, polymer induced crystallization, and grinding.

In some embodiments, crystalline Type A of Formula I can be made by a process comprising:
dissolving N-(3,5-difluorobenzyl)-N-hydroxy-2,2-dimethylbutanamide in a first solvent to obtain a solution;
adding a second solvent into the solution to obtain a suspension while stirring the solution;
isolating a solid from the suspension; and drying the solid to provide crystalline Type A of Formula I.

In certain embodiments, the first solvent is a solvent in which N-(3,5-difluorobenzyl)-N-hydroxy-2,2-dimethylbutanamide has a solubility of >10 mg/ml, such as >15 mg/ml, >20 mg/ml, >25 mg/ml, >30 mg/ml, or >35 mg/ml; and the second solvent is a solvent in which N-(3,5-difluorobenzyl)-N-hydroxy-2,2-dimethylbutanamide has a solubility of <10 mg/ml, such as <8 mg/ml, <6 mg/ml, <4 mg/ml, <3 mg/ml, <2 mg/ml, or <1 mg/ml.

In certain embodiments, the first solvent is selected from the group consisting of isoamyl alcohol, ethyl lactate, MEK, anisole, n-butanol/n-BuOH, ethyl formate, 2,2,2-trifluoroethanol, toluene, pyridine, isobutanol, chlorobenzene, CPME, m-xylene, n-butyl acetate, cumene, NMP, MTBE, 2-MeTHF, EtOAc, Acetone, THF, DMAc, IPA, EtOH, DCM, MeOH, IPA, MIBK, IPAc, THF, 1,4-dioxane, DCM, CHCl₃, toluene, DMSO, DMAc, NMP, and ACN; and the second solvent is selected from the group consisting of n-hexane, water, 2-Me THF, MTBE, cyclohexane, and n-heptane.

In certain embodiments, the first solvent is selected from the group consisting of EtOAc, Acetone, THF, DMAc, IPA, EtOH, DCM, and MeOH; and the second solvent is selected from the group consisting of water, 2-Me THF, cyclohexane, and n-heptane.

### Type C of Formula I

In some embodiments, crystalline Type C of Formula I has an XRPD pattern substantially the same as one of the XRPD patterns indicated for crystalline Type C of N-(3,5-difluorobenzyl)-N-hydroxy-2,2-dimethylbutanamide shown in FIG. 47 and FIG. 48.

In some embodiments, crystalline Type C of Formula I has an XRPD pattern characterized by at least two, at least three, at least four, at least five, or at least six of the degree 2θ-peaks with the greatest intensity as substantially shown in one of the XRPD patterns indicated for crystalline Type C of N-(3,5-difluorobenzyl)-N-hydroxy-2,2-dimethylbutanamide shown in FIG. 47 and FIG. 48.

Crystalline Type C of Formula I can be converted to Type A after drying.

In some embodiments, crystalline Type C of Formula I has an XRPD pattern comprising degree 2θ-peaks (±0.2 degrees 2θ) at 21.78 and one of the degree 20-peaks (±0.2 degrees 2θ) at 9.08, 13.45, 18.18, and 27.39.

In some embodiments, crystalline Type C of Formula I has an XRPD pattern comprising degree 2θ-peaks (±0.2 degrees 2θ) at 21.78 and two of the degree 20-peaks (±0.2 degrees 2θ) at 9.08, 13.45, 14.28, 18.18, and 27.39.

In some embodiments, crystalline Type C of Formula I has an XRPD pattern comprising degree 2θ-peaks (±0.2 degrees 2θ) at 21.78 and three of the degree 20-peaks (±0.2 degrees 2θ) at 9.08, 13.45, 14.28, 18.18, and 27.39.

In some embodiments, crystalline Type C of Formula I has an XRPD pattern comprising degree 20 (2 theta)-peaks (±0.2 degrees 2θ) at 9.08, 18.18, and 21.78.

In some embodiments, crystalline Type C of Formula I has an XRPD pattern comprising degree 2θ-peaks (±0.2 degrees 2θ) at 9.08, 18.18, 21.78, and 27.39. In some embodiments, crystalline Type C of Formula I has an XRPD pattern comprising degree 2θ-peaks (±0.2 degrees 2θ) at 9.08, 13.45, 14.28, 18.18, 21.78, and 27.39. In some embodiments, crystalline Type C of Formula I has an XRPD pattern comprising degree 2θ-peaks (±0.2 degrees 2θ) at 9.08, 13.45, 13.91, 14.28, 18.18, 21.78, 27.39, 30.80, 36.77, and 37.14. In some embodiments, crystalline Type C of Formula I has an XRPD pattern comprising any three degree 2θ-peaks (±0.2 degrees 2θ) selected from the group consisting of 9.08, 13.45, 14.28, 18.18, 21.78, and 27.39.

In one embodiment, crystalline Type C of Formula I comprises the XRPD peaks substantially shown below in Table 3.

TABLE 3

| Peak Position (degree 2θ) | Relative Intensity (%) |
|---|---|
| 9.08 | 55.35 |
| 13.45 | 12.97 |
| 13.91 | 6.48 |
| 14.28 | 12.00 |
| 17.24 | 4.52 |
| 18.18 | 55.22 |
| 21.78 | 100.00 |
| 25.98 | 4.93 |
| 26.32 | 3.73 |
| 27.39 | 16.57 |
| 30.80 | 7.61 |
| 36.26 | 2.71 |
| 36.77 | 6.81 |
| 37.14 | 8.45 |

### III. Compositions

Another aspect of the disclosure provides a pharmaceutical composition comprising a crystalline form (e.g., Type A or Type C) of Formula I and at least one pharmaceutically acceptable carrier. In some embodiments, the crystalline form (e.g., Type A or Type C) of Formula I in a pharmaceutical composition is substantially free of other solid forms of Formula I.

In some embodiments, the pharmaceutically acceptable carrier is selected from pharmaceutically acceptable vehicles and pharmaceutically acceptable adjuvants. In some embodiments, the pharmaceutically acceptable carrier is chosen from pharmaceutically acceptable fillers, disintegrants, surfactants, binders, and lubricants.

It will also be appreciated that a pharmaceutical composition of this disclosure can be employed in combination therapies; that is, the pharmaceutical compositions described herein can further include an additional active pharmaceutical agent. Alternatively, a pharmaceutical composition comprising a substantially pure crystalline form (e.g., Type A or Type C) of Formula I can be administered as a separate composition concurrently with, prior to, or subsequent to, a composition comprising an additional active pharmaceutical agent.

In some embodiments, the pharmaceutically acceptable carrier may be chosen from adjuvants and vehicles. The pharmaceutically acceptable carrier, as used herein, can be chosen, for example, from any and all solvents, diluents, other liquid vehicles, dispersion aids, suspension aids, surface active agents, isotonic agents, thickening agents, emulsifying agents, preservatives, solid binders, and lubricants, which are suited to the particular dosage form desired. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988 to 1999, Marcel Dekker, New York discloses various carriers used in formulating pharmaceutical compositions and known techniques for the preparation thereof. Except insofar as any conventional carrier is incompatible with the compounds of this disclosure, such as by producing any undesirable biological effect or otherwise interacting in a deleterious manner with any other component(s) of the pharmaceutical composition, its use is contemplated to be within the scope of this disclosure. Non-limiting examples of suitable pharmaceutically acceptable carriers include ion exchangers, alumina, aluminum stearate, lecithin, serum proteins (such as human serum albumin), buffer substances (such as phosphates, glycine, sorbic acid, and potassium sorbate), partial glyceride mixtures of saturated vegetable fatty acids, water, salts, and electrolytes (such as protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as lactose, glucose and sucrose), starches (such as corn starch and potato starch), cellulose and its derivatives (such as sodium carboxymethyl cellulose, ethyl cellulose and cellulose acetate), powdered tragacanth, malt, gelatin, talc, excipients (such as cocoa butter and suppository waxes), oils (such as peanut oil, cottonseed oil, safflower oil, sesame oil, olive oil, corn oil and soybean oil), glycols (such as propylene glycol and polyethylene glycol), esters (such as ethyl oleate and ethyl laurate), agar, buffering agents (such as magnesium hydroxide and aluminum hydroxide), alginic acid, pyrogen-free water, isotonic saline, Ringer's solution, ethyl alcohol, phosphate buffer solutions, non-toxic compatible lubricants (such as sodium lauryl sulfate and magnesium stearate), coloring agents, releasing agents, coating agents, sweetening agents, flavoring agents, perfuming agents, preservatives, and antioxidants.

A substantially pure crystalline form (e.g., Type A or Type C) of Formula I or a pharmaceutical composition disclosed herein can be administered orally in solid dosage forms, such as capsules, tablets, troches, dragées, granules and powders, or in liquid dosage forms, such as elixirs, syrups, emulsions, dispersions, and suspensions. The crystalline form of Formula I described herein can also be administered parenterally, in sterile liquid dosage forms, such as dispersions, suspensions or solutions. Other dosages forms that can also be used to administer the crystalline form of Formula I described herein as an ointment, cream, drops, transdermal patch or powder for topical administration, as an ophthalmic solution or suspension formation, e.g., eye drops, for ocular administration, as an aerosol spray or powder composition for inhalation or intranasal administration, or as a cream, ointment, spray or suppository for rectal or vaginal administration.

Gelatin capsules containing a crystalline form of Formula I disclosed herein and powdered carriers, such as lactose, starch, cellulose derivatives, magnesium stearate, stearic acid, and the like, can also be used. Similar diluents can be used to make compressed tablets. Both tablets and capsules can be manufactured as sustained release products to provide for continuous release of medication over a period of time. Compressed tablets can be sugar coated or film coated to mask any unpleasant taste and protect the tablet from the atmosphere, or enteric coated for selective disintegration in the gastrointestinal tract.

Liquid dosage forms for oral administration can further comprise at least one agent selected from coloring and flavoring agents to increase patient acceptance.

In general, water, a suitable oil, saline, aqueous dextrose (glucose), and related sugar solutions and glycols such as propylene glycol or polyethylene glycols can be examples of suitable carriers for parenteral solutions. Solutions for parenteral administration may comprise a water soluble salt of the at least one compound describe herein, at least one suitable stabilizing agent, and if necessary, at least one buffer substance. Antioxidizing agents such as sodium bisulfite, sodium sulfite, or ascorbic acid, either alone or combined, can be examples of suitable stabilizing agents. Citric acid and its salts and sodium EDTA can also be used as examples of suitable stabilizing agents. In addition, parenteral solutions can further comprise at least one preservative, selected, for example, from benzalkonium chloride, methyl- and propylparaben, and chlorobutanol.

A pharmaceutically acceptable carrier is, for example, selected from carriers that are compatible with active ingredients of the composition (and in some embodiments, capable of stabilizing the active ingredients) and not deleterious to the subject to be treated. For example, solubilizing agents, such as cyclodextrins (which can form specific, more soluble complexes with the at least one compound and/or at least one pharmaceutically acceptable salt disclosed herein), can be utilized as pharmaceutical excipients for delivery of the active ingredients. Examples of other carriers include colloidal silicon dioxide, magnesium stearate, cellulose, sodium lauryl sulfate, and pigments such as D&C Yellow #10. Suitable pharmaceutically acceptable carriers are described in Remington's Pharmaceutical Sciences, A. Osol.

For administration by inhalation, a crystalline form of Formula I described herein may be conveniently delivered in the form of an aerosol spray presentation from pressurized packs or nebulisers. A crystalline form of Formula I described herein may also be delivered as powders, which may be formulated, and the powder composition may be inhaled with the aid of an insufflation powder inhaler device. One exemplary delivery system for inhalation can be metered dose inhalation (MDI) aerosol, which may be formulated as a suspension or solution of a crystalline form described herein in at least one suitable propellant, selected, for example, from fluorocarbons and hydrocarbons.

For ocular administration, an ophthalmic preparation may be formulated with an appropriate weight percentage of a solution or suspension of a crystalline form of Formula I, described herein in an appropriate ophthalmic vehicle, such that the crystalline form of Formula I described herein is maintained in contact with the ocular surface for a sufficient time period to allow the active compound to penetrate the corneal and internal regions of the eye.

Useful pharmaceutical dosage-forms for administration of a crystalline form of Formula I described herein include, but are not limited to, hard and soft gelatin capsules, tablets, parenteral injectables, and oral suspensions. In some embodiments, the pharmaceutical compositions disclosed herein may be in the form of controlled release or sustained release compositions as known in the art.

The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules, lozenges or the like in the case of solid compositions. In such compositions, the active material is usually a component ranging from about 0.1 to about 50% by weight or preferably from about 1 to about 40% by weight with the remainder being various vehicles or carriers and processing aids helpful for forming the desired dosing form. Unit dosage formulations are preferably about of 5, 10, 25, 50, 100, 250, 500, or 1,000 mg per unit. In a particular embodiment, unit dosage forms are packaged in a multipack adapted for sequential use, such as blisterpack comprising sheets of at least 6, 9 or 12 unit dosage forms.

In some embodiments, unit capsules can be prepared by filling standard two-piece hard gelatin capsules each with, for example, 100 milligrams of a crystalline form of Formula I described herein in powder, 150 milligrams of lactose, 50 milligrams of cellulose, and 6 milligrams magnesium stearate.

In some embodiments, a mixture of a crystalline form of Formula I described herein and a digestible oil such as soybean oil, cottonseed oil or olive oil can be prepared and injected by means of a positive displacement pump into gelatin to form soft gelatin capsules containing 100 milligrams of the active ingredient. The capsules are washed and dried.

In some embodiments, tablets can be prepared by conventional procedures so that the dosage unit comprises, for example, 100 milligrams of a crystalline form of Formula I, or pharmaceutically acceptable salts thereof, 0.2 milligrams of colloidal silicon dioxide, 5 milligrams of magnesium stearate, 275 milligrams of microcrystalline cellulose, 11 milligrams of starch and 98.8 milligrams of lactose. Appropriate coatings may be applied to increase palatability or delay absorption.

In some embodiments, a parenteral composition suitable for administration by injection can be prepared by stirring 1.5% by weight of a crystalline form of Formula I and/or at least an enantiomer, or pharmaceutically acceptable salt thereof disclosed herein in 10% by volume propylene glycol. The solution is made to the expected volume with water for injection and sterilized.

In some embodiment, an aqueous suspension can be prepared for oral administration. For example, each 5 milliliters of an aqueous suspension comprising 100 milligrams of finely divided compound, or pharmaceutically acceptable salts thereof, 100 milligrams of sodium carboxymethyl cellulose, 5 milligrams of sodium benzoate, 1.0 grams of sorbitol solution, U.S.P., and 0.025 milliliters of vanillin can be used.

The same dosage forms can generally be used when a crystalline form of Formula I described herein is administered stepwise or in conjunction with at least one other therapeutic agent. When drugs are administered in physical combination, the dosage Type And administration route should be selected depending on the compatibility of the combined drugs. Thus, the term coadministration is understood to include the administration of at least two agents concomitantly or sequentially, or alternatively as a fixed dose combination of the at least two active components.

A crystalline form of Formula I disclosed herein can be administered as the sole active ingredient or in combination with at least one second active ingredient.

A crystalline form (e.g., Type A or Type C) of Formula I described herein may be used per se, or in the form of their pharmaceutically acceptable salts, such as hydrochlorides, hydrobromides, acetates, sulfates, citrates, carbonates, trifluoroacetates and the like. Salts can be obtained by addition of the desired base, either neat or in a suitable inert solvent. Examples of pharmaceutically acceptable base addition salts include sodium, potassium, calcium, ammonium, organic amino, or magnesium salts, or the like. Salts can also be obtained by addition of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like (see, for example, Berge et al., “Pharmaceutical Salts,” Journal of Pharmaceutical Science, 1977, 66, 1-19).

### IV. Methods of Treatment and Uses

Another aspect of the disclosure provides a method of treating a disease or condition, comprising administering to a subject in need thereof, a therapeutically effective amount of a substantially pure crystalline form (e.g., Type A or Type C) of Formula I or a pharmaceutical composition thereof, wherein the disease or condition is selected from an inflammatory disease, an immune disease (e.g., an autoimmune disease), an allergic disease, transplant rejection, a necrotic cell disease, a neurodegenerative disease, a CNS disease, ischemic brain injury, an ocular disease, an infectious disease, a malignancy, ulcerative colitis, Crohn's disease, psoriasis, rheumatoid arthritis, ALS, Alzheimer's disease, and a viral infection. In some embodiments, the disease or condition is mediated by RIP1 signaling.

In another aspect, disclosed herein is a substantially pure crystalline form (e.g., Type A or Type C) of Formula I or a pharmaceutical composition thereof, for use as a medicament.

In another aspect, disclosed herein is use of a substantially pure crystalline form (e.g., Type A or Type C) of Formula I or a pharmaceutical composition thereof, for the manufacture of a medicament for treating a disease or condition selected from an inflammatory disease, an immune disease (e.g., an autoimmune disease), an allergic disease, transplant rejection, a necrotic cell disease, a neurodegenerative disease, a CNS disease, ischemic brain injury, an ocular disease, an infectious disease, a malignancy, ulcerative colitis, Crohn's disease, psoriasis, rheumatoid arthritis, ALS, Alzheimer's disease, and a viral infection. In some embodiments, the disease or condition is mediated by RIP1 signaling.

In a further aspect of this disclosure, a substantially pure crystalline form (e.g., Type A or Type C) of Formula I or a pharmaceutical composition thereof, is for use in treating a disease or condition selected from an inflammatory disease, an immune disease (e.g., an autoimmune disease), an allergic disease, transplant rejection, a necrotic cell disease, a neurodegenerative disease, a CNS disease, ischemic brain injury, an ocular disease, an infectious disease, a malignancy, ulcerative colitis, Crohn's disease, psoriasis, rheumatoid arthritis, ALS, Alzheimer's disease, and a viral infection. In some embodiments, the disease or condition is mediated by RIP1 signaling.

Another aspect of the disclosure provides a method of modulating, e.g., inhibiting, RIP1 signaling in a subject in need thereof, comprising administering to the subject, a therapeutically effective amount of a substantially pure crystalline form (e.g., Type A or Type C) of Formula I or a pharmaceutical composition thereof.

In another aspect, disclosed herein is use of a substantially pure crystalline form (e.g., Type A or Type C) of Formula I or a pharmaceutical thereof, for modulating, e.g., inhibiting, RIP1 signaling in a subject in need thereof.

In another aspect of this disclosure, a substantially pure crystalline form (e.g., Type A or Type C) of Formula I or a pharmaceutical composition thereof, is for use in modulating, e.g., inhibiting, RIP1 signaling in a subject in need thereof by contacting the subject with the crystalline form, or the pharmaceutical composition.

A compound of the Formulae disclosed herein, a substantially pure crystalline form (e.g., Type A or Type C) of Formula I or a pharmaceutical composition thereof may be administered once daily, twice daily, or three times daily, for example, for the treatment of a disease or condition, selected from an inflammatory disease, an immune disease (e.g., an autoimmune disease), an allergic disease, transplant rejection, a necrotic cell disease, a neurodegenerative disease, a CNS disease, ischemic brain injury, an ocular disease, an infectious disease, a malignancy, ulcerative colitis, Crohn's disease, psoriasis, rheumatoid arthritis, ALS, Alzheimer's disease, and a viral infection. In some embodiments, the disease or condition is mediated by RIP1 signaling.

A substantially pure crystalline form (e.g., Type A or Type C) of Formula I or a pharmaceutical composition thereof may be administered, for example, in various manners, such as orally, topically, rectally, parenterally, by inhalation spray, or via an implanted reservoir, although the most suitable route in any given case will depend on the particular host, and nature and severity of the conditions for which the active ingredient is being administered. The term “parenteral” as used herein includes subcutaneous, intracutaneous, intravenous, intramuscular, intraarticular, intraarterial, intrasynovial, intrasternal, intrathecal, intralesional and intracranial injection or infusion techniques. The compositions disclosed herein may be conveniently presented in unit dosage form and prepared by any of the methods well known in the art. Parenteral administration can be by continuous infusion over a selected period of time. Other forms of administration contemplated in this disclosure are as described in International Patent Application Nos. WO 2013/075083, WO 2013/075084, WO 2013/078320, WO 2013/120104, WO 2014/124418, WO 2014/151142, and WO 2015/023915.

The contacting is generally effected by administering to the subject an effective amount of a crystalline form of Formula I disclosed herein. Generally, administration is adjusted to achieve a therapeutic dosage of about 0.1 to 50, preferably 0.5 to 10, more preferably 1 to 10 mg/kg, though optimal dosages are compound specific, and generally empirically determined for each compound.

The dosage administered will be dependent on factors, such as the age, health and weight of the recipient, the extent of disease, type of concurrent treatment, if any, frequency of treatment, and the nature of the effect desired. In general, a daily dosage of the active ingredient can vary, for example, from 0.1 to 2000 milligrams per day. For example, 10-500 milligrams once or multiple times per day may be effective to obtain the desired results.

In some embodiments, 2 mg to 1500 mg or 5 mg to 1000 mg of a compound of the Formulae disclosed herein, a crystalline form of Formula I or a pharmaceutical composition thereof are administered once daily, twice daily, or three times daily. A crystalline form of Formula I described herein is administered for morning/daytime dosing, with off period at night.

### Examples

The following examples are provided to describe the disclosure in greater detail. They are intended to illustrate, not to limit, the disclosure.

### Example 1: Synthesis of N-(3,5-difluorobenzyl)-N-hydroxy-2,2-dimethylbutanamide

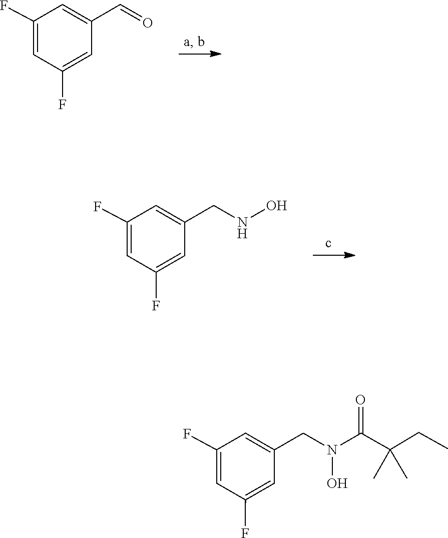 [SMILES: O=Cc1cc(F)cc(F)c1 ; ONCc1cc(F)cc(F)c1 ; CCC(C)(C)C(=O)N(O)Cc1cc(F)cc(F)c1]

Reagents and conditions: (a) NH₂OH·HCl, Na₂CO₃; (b) BH₃·Py; (c) 2,2-dimethylbutanoyl chloride, NaHCO₃, THF/H₂O, 0° C. for 30 min, rt for 16 h

A mixture of 3,5-difluorobenzaldehyde (400 mg, 2.81 mmol) and hydroxylamine hydrochloride (215.15 mg, 3.10 mmol, 1.1 equiv.) was stirred at room temperature in a solution (THF/EtOH/H2O, 4 mL/10 mL/2 mL) for 16 h. The mixture was extracted with EtOAc, washed with water and brine, dried (over Na₂SO₄), and concentrated in vacuo to give 3,5-difluorobenzaldehyde oxime as a white solid, which was used for the next step without further purification.

A mixture of 3,5-difluorobenzaldehyde oxime and 8M pyridine-borane complex (0.64 mL) in 5 mL EtOH and 2 mL THF was kept below 5° C. 10% HCl (6.5 mL) was added to the mixture dropwise. The mixture was then warmed up for 30 min to room temperature. The mixture was neutralized with Na₂CO₃, extracted with EtOAc, washed with water and brine, dried (over Na₂SO₄), and concentrated in vacuo to give a crude product of N-(3,5-difluorobenzyl)hydroxylamine, which was used directly in the next step without purification.

N-(3,5-difluorobenzyl)hydroxylamine was dissolved in 2 mL of THF/H₂O (1:1) and 0.44 mL of saturated aqueous NaHCO₃. The solution was cooled to 0° C. and 2,2-dimethylbutanoylchloride (92 mg) was added and the mixture was stirred at room temperature for 16 h. The mixture was extracted with EtOAc for three times and the combined organic layers were washed with brine, dried (over Na₂SO₄) and concentrated in vacuo. Purification by silica gel chromatography gave the title compound (311 mg) in a 43% total yield. ¹HNMR (400 MHz, DMSO-d6) δ9.76 (s, 1H), 7.09 (td, J=9.4, 2.1 Hz, 1H), 6.95-6.86 (m, 2H), 4.66 (s, 2H), 1.64 (q, J=7.5 Hz, 2H), 1.13 (s, 6H), 0.72 (t, J=7.5 Hz, 3H). LC-MS (m/z) 258.4 (M+H+).

Crystalline forms of the title compound were obtained as disclosed herein. Certain batches of the title compound, e.g., in crystalline Type A, were micronized to reduce the particle size. The compound was charged into a feeder hopper, and then charged into a jet mill. The milling was started and continued until the desired API particle size (D90) was obtained, e.g., at about 30-40 μm or about 15 μm. In some batches, the API particle size (D90) was not more than 20 μm.

### Example 2: Preparation of Crystalline Type A of N-(3,5-difluorobenzyl)-N-hydroxy-2,2-dimethylbutanamide by Anti-Solvent Addition and Reverse Anti-Solvent Addition

Two anti-solvent addition experiments were carried out. About 15 mg of N-(3,5-difluorobenzyl)-N-hydroxy-2,2-dimethylbutanamide (“starting material”) (810048-48-B) was dissolved in 0.1˜0.2 mL of a solvent as shown in Table 4 to obtain a clear solution. The solution was magnetically stirred with addition of an anti-solvent till precipitates appeared or the total volume of anti-solvent reached 10 mL. The obtained precipitates were isolated for XRPD analysis. XRPD patterns are displayed in FIG. 7, showing only Type A was generated. A DSC curve in FIG. 8 showing only a sharp endotherm at 95.1° C. (onset) was observed for Type A sample (810048-19-B2).

TABLE 4

| Experiment ID | Solvent | Anti-solvent | Result |
|---|---|---|---|
| 810048-19-B1 | EtOAc | n-Heptane | Type A |
| 810048-19-B2 | CHCl₃ | n-Heptane | Type A |

Another 12 anti-solvent addition experiments were carried out. About 15 mg of a starting material (817506-01-A) was dissolved in 0.1˜0.2 mL of a solvent as shown in Table 5 to obtain a clear solution. The solution was magnetically stirred with addition of an anti-solvent till precipitates appeared or the total volume of anti-solvent reached 10 mL. The obtained precipitates were isolated for XRPD analysis. Results in Table 5 showed that Type A and a mixture of Type A+Impurity B were generated using the different sets of solvents and anti-solvents.

TABLE 5

| Experiment ID | Solvent | Anti-solvent | Result |
|---|---|---|---|
| 817506-04-A1 | MeOH | H₂O | Type A |
| 817506-04-A2 | Acetone | | Type A |
| 817506-04-A3 | THF | | Type A |
| 817506-04-A4 | DMAc | | Type A |
| 817506-04-A5* | IPA | n-Heptane | Type A |
| 817506-04-A6 | MIBK | | Impurity B |
| 817506-04-A7 | EtOAc | | Type A + Impurity B |
| 817506-04-A8 | CHCl₃ | | Type A + Impurity B |
| 817506-04-A9* | EtOH | 2-MeTHF | Type A |
| 817506-04-A10* | DCM | | Type A |
| 817506-04-A11 | Acetone | Cyclohexane | Type A |
| 817506-04-A12 | DCM | | Type A |

*Clear solutions were obtained after anti-solvent addition and slurry at 5° C., which were transferred to evaporate at RT.

About 20 mg of starting material (8152100-01-A) was added into a 20-mL glass vial and dissolved in 0.2˜2.0 mL corresponding solvent to obtain a clear solution. The solution was magnetically stirred with addition of anti-solvent till precipitates appeared or the total volume of anti-solvent reached 3 mL. The obtained precipitates were isolated for XRPD analysis. If solids were not obtained, slurry at 5° C./−20° C. or evaporation at RT was performed. Results in Table 6 showed that Type A was generated.

TABLE 6

| Experiment ID | Solvent | Anti-solvent | Result |
|---|---|---|---|
| 8152100-04-A1 | IPA | H₂O | Type A |
| 8152100-04-A2 | ACN | | Type A |
| 8152100-04-A3 | 1,4-Dioxane | | Type A |
| 8152100-04-A5 | Isoamyl alcohol | n-Heptane | Type A^{#} |
| 8152100-04-A6 | Ethyl lactate | | Type A* |
| 8152100-04-A7 | MEK | | Type A |
| 8152100-04-A8 | Anisole | | Type A |
| 8152100-04-A9 | n-Butanol | Cyclohexane | Type A^{#} |
| 8152100-04-A10 | MIBK | | Type A |
| 8152100-04-A11 | Ethyl formate | | Type A |
| 8152100-04-A12 | 2,2,2-Trifluoroethanol | | Type A* |

*Oil-like sample was obtained after anti-solvent addition and slurry with temperature cycling between 50~5° C. (3 cycles), which was transferred to evaporation at RT.
^{#}Clear solution was obtained after anti-solvent addition and slurry at 5° C./−20° C., which was transferred to evaporation at RT

One reverse anti-solvent addition experiment was carried out. About 20 mg of starting material (8152100-01-A) was added into a 5-mL glass vial and dissolved in 0.2˜2.0 mL corresponding solvent to obtain a clear API solution. Then, 5 mL anti-solvent in Table 7 was added into a 20-mL glass vial. The API solution was added into the 20-mL vial containing the anti-solvent with magnetic stirring.

The obtained precipitates were isolated for XRPD analysis. If solids were not obtained after addition of API solution, slurry at 5° C./—20° C. or evaporation at RT was performed. Results in Table 7 showed that Type A was generated.

TABLE 7

| Experiment ID | Solvent | Anti-solvent | Result |
|---|---|---|---|
| 8152100-05-A1 | Toluene | n-Hexane | Type A |
| 8152100-05-A2 | IPA | | Type A\hat{ } |
| 8152100-05-A3 | Pyridine | | Type A* |
| 8152100-05-A4 | CHCl₃ | | Type A |
| 8152100-08-A5 | EtOAc | | Type A |
| 8152100-05-A6 | Isobutanol | Cyclohexane | Type A\hat{ } |
| 8152100-05-A7 | Chlorobenzene | | Type A |
| 8152100-05-A8 | CPME | | Type A |
| 8152100-05-A9 | Ethyl formate | | Type A |
| 8152100-0S-A10 | EtOH | n-Heptane | Type A^{#} |
| 8152100-05-A11 | m-Xylene | | Type A |
| 8152100-05-A12 | n-Butyl acetate | | Type A |
| 8152100-05-A13 | Cumene | | Type A |
| 8152100-05-A14 | NMP | H₂O | Type A |
| 8152100-05-A15 | MeOH | | Type A |
| 8152100-05-A16 | Acetone | | Type A |
| 8152100-05-A17 | THF | | Type A |
| 8152100-05-A18 | EtOAc | | Type A |
| 8152100-22-A1 | n-BuOH | n-Hexane | Type A** |
| 8152100-22-A2 | Ethyl formate | | Type A |
| 8152100-22-A3 | MIBK | | Type A |
| 8152100-22-A4 | m-Xylene | | Type A |
| 8152100-22-A5 | EtOAc | Cyclohexane | Type A |
| 8152100-22-A6 | MTBE | | Type A |
| 8152100-22-A7 | CHCl₃ | | Type A |
| 8152100-22-A8 | MEK | | Type A** |
| 8152100-22-A9 | Acetone | n-Heptane | Type A |
| 8152100-22-A10 | IPAc | | Type A |
| 8152100-22-A11 | 2-MeTHF | | Type A |
| 8152100-22-A12 | Pyridine | | Type A** |
| 8152100-22-A13 | EtOH | H₂O | Type A |
| 8152100-22-A14 | ACN | | Type A |
| 8152100-22-A15 | 1,4-Dioxane | | Type A |
| 8152100-22-A16 | 2,2,2-Influoroethanol | | Type A |

*Clear solution was obtained after reverse anti-solvent addition and slurry at 5° C./−20° C., so the sample was then transferred to evaporation at RT.
^{#}No solid was obtained after reverse anti-solvent addition. Solid was obtained after slurry at 5° C.
\hat{ }Clear solution was obtained after reverse anti-solvent addition and slurry at 5° C./−20° C. so the sample was transferred to evaporation at RT. Compared with the pattern of Type A, a few extra weak diffraction peaks were observed, the intensity of which was slightly stronger than background noise. Since very limited amount of sample was obtained, no further research was performed.
**Clear solution was obtained after reverse anti-solvent addition and slurry at 5° C. for 2 days, so the sample was transferred to evaporation at RT.

### Example 3: Preparation of Crystalline Type A of N-(3,5-difluorobenzyl)-N-hydroxy-2,2-dimethylbutanamide by Solid Vapor Diffusion

Solid vapor diffusion experiments were conducted using five different solvents as shown in Table 8. Approximately 12 mg of a starting material (810055-01-A) was weighed into a 3-mL vial, which was placed into a 20-mL vial with 4 mL of a volatile solvent. The 20-mL vial was sealed with a cap and kept at RT (room temperature) for 7 days allowing solvent vapor to interact with the sample. The dissolved sample in 3-ml vial was taken out for evaporation at RT to obtain a solid. The solids were tested by XRPD and the results summarized in Table 8 showed that only Type A was obtained. XRPD and DSC results are shown in FIG. 9 and FIG. 10. Only a sharp endotherm at about 95° C. (onset) was observed on the DSC curves in FIG. 10.

TABLE 8

| Experiment ID | Solvent | Result |
|---|---|---|
| 810053-02-A1 | H₂O | Type A |
| 810055-02-A2* | IPA | Type A |
| 810055-02-A3* | CHCl₃ | Type A |
| 810055-02-A4* | Acetone | Type A |
| 810055-02-A5* | IPAc | Type A |

*Clear solutions were obtained after 7 days, which were transferred to evaporate at RT.

Additional solid vapor diffusion experiments were conducted using 12 different solvents as shown in Table 9. Approximately 12 mg of a starting material (817506-01-A) was weighed into a 3-mL vial, which was placed into a 20-mL vial with 4 mL of a volatile solvent. The 20-mL vial was sealed with a cap and kept at RT for 7 days allowing solvent vapor to interact with the sample. The solids were tested by XRPD and the results summarized in Table 9 showed that only Type A and a mixture of Type A+Impurity B were obtained with the different solvents used.

TABLE 9

| Experiment ID | Solvent | Result |
|---|---|---|
| 817506-05-A1 | H₂O | Type A + Impurity B |
| 817506-05-A2* | IPA | Type A + Impurity B |
| 817506-05-A3* | CHCl₃ | Type A + Impurity B |
| 817506-05-A4* | EtOH | Type A |
| 817506-05-A5* | MeOH | Type A |
| 817506-05-A6* | ACN | Type A |
| 817506-05-A7* | THF | Type A |
| 817506-05-A8* | 1,4-Dioxane | Type A |
| 817506-05-A9* | Acetone | Type A + Impurity B |
| 817506-05-A10* | MIBK | Type A |
| 817506-05-A11* | EtOAc | Type A |
| 817506-05-A12* | IPAC | Type A + Impurity B |

*Clear solutions were obtained after 7 days, which were transferred to evaporate at RT.

### Example 4: Preparation of Crystalline Type A of N-(3,5-difluorobenzyl)-N-hydroxy-2,2-dimethylbutanamide by Slurry at RT

Slurry conversion experiments were conducted at RT in different solvent systems. About 20 mg of a starting material (810055-01-A) was suspended in 0.2˜0.4 mL of a solvent as shown in Table 10 in an HPLC vial. After the suspension was stirred magnetically for 4 days at RT, the remaining solids were isolated for XRPD analysis. Results summarized in Table 10 indicated that only Type A was generated. XRPD and DSC results are displayed from FIG. 11 to FIG. 14.

TABLE 10

| Experiment ID | Solvent (v/v) | Result |
|---|---|---|
| 810055-03-A1* | IPA | Type A |
| 810055-03-A2* | MIBK | Type A |
| 810055-03-A3* | MTBE | Type A |
| 810055-03-A4* | 2-MeTHF | Type A |
| 810055-03-A5 | n-Heptane | Type A |
| 810055-03-A6* | EtOH/toluene (1:1) | Type A |
| 810055-03-A7* | MIBK/MTBE (1:1) | Type A |
| $10055-03-A8* | Acetone/1,4-dioxane (1:1) | Type A |
| 810055-03-A9* | IPAc/cyclohexane (1:1) | Type A |
| 810055-03-A10* | IPA/H₂O (982:18, a_{w}~0.2) | Type A |
| 810055-03-A11* | IPAH₂O (956:44, a_{w}~0.4) | Type A |

*Clear solution was obtained after slurry at RT for 3 days, and then at 5° C. for another 2 days, which was transferred to evaporate at RT.

Additional slurry conversion experiments were conducted at RT in different solvent systems. About 20˜40 mg of a starting material (817506-01-A) was suspended in 0.2˜0.4 mL of a solvent as shown in Table 11 in an HPLC vial. After the suspension was stirred magnetically for 4 days at RT, the remaining solids were isolated for XRPD analysis. Results summarized in Table 11 indicated that Type A, a mixture of Type A+Impurity B, and an oil-like sample were generated using the different solvent systems.

TABLE 11

| Experiment ID | Solvent (v/v) | Result |
|---|---|---|
| 817506-06-A1^{&} | EtOH | Type A |
| 817506-06-A2^{&} | IPA | Type A + Impurity B |
| 817506-06-A3^{&} | Acetone | Type A |
| 817506-06-A4^{&} | MIBK | Type A + Impurity B |
| 817506-06-A5 | MTBE | Type A + Impurity B |
| 817506-06-A6^{&} | THF | Oil |
| 817506-06-A7 | 2-MeTHF | Type A + Impurity B |
| 817506-06-A8^{#} | 1,4-Dioxane | Type A |
| 817506-06-A9 | n-Heptane | Type A + Impurity B |
| 817506-06-A10* | Toluene | Type A |
| 817506-06-A11 | H₂O | Type A |
| 817506-06-A12^{&} * | EtOH/toluene (1:1) | Type A + Impurity B |
| 817506-06-A13^{&} | MeOH/2-MeTHF (1:1) | Oil |
| 817506-06-A14^{&} | MIBK/MTBE (1:1) | Type A + Impurity B |
| 817506-06-A15^{#} | EtOAc/n-heptane (1:1) | Type A |
| 817506-06-A16 | DMSO/H₂O (1:9) | Type A |
| 817506-06-A17 | DCM/n-heptane (1:4) | Type A |
| 817506-06-A18^{#} | ACN/toluene (1:9) | Type A |
| 817506-06-A19^{&} | Acetone/1,4-dioxane (1:1) | Type A + Impurity B |
| 817506-06-A20^{#} | IPAc/cyclohexane (1:1) | Type A + Impurity B |
| 817506-06-A21^{&} | IPA/H₂O (982:18, a_{w}~0.2) | Type A + Impurity B |
| 817506-06-A22^{&} | IPAH₂O (956:44, a_{w}~0.4) | Type A + Impurity B |
| 817506-06-A23^{#} | IPA/H₂O (919:81, a_{w}~0.6) | Type A |
| 817506-06-A24^{#} | IPA/H₂O (850:150, a_{w}~0.8) | Type A |

^{#}Clear solutions were obtained after slurry at RT for 3 days followed by between 50° C. and 5° C. (3 cycles), which were transferred to slurry at −20° C. to obtain solids.
^{&}Clear solutions were obtained after slurry at RT for 3 days and between 50° C. and 5° C. (3 cycles), followed by slurry at −20° C. Add anti-solvent H₂O or n-heptane and slurry at RT for 3 days.
*Clear solution was obtained after adding anti-solvent n-heptane, which was transferred to evaporate at RT.

### Example 5: Preparation of Crystalline Type A of N(3,5-difluorobenzyl)-N-hydroxy-2,2-dimethylbutanamide by Slurry at 50° C. and −20° C. and Slurry with Temperature Cycling

Slurry conversion experiments were conducted at 50° C. in different solvent systems. About 25 mg of a starting material (810055-01-A) was suspended in 0.2˜0.4 mL of a solvent as shown in Table 12 in an HPLC vial. After the suspension was magnetically stirred for about 3 days at 50° C., the remaining solids were isolated for XRPD analysis. Results summarized in Table 12 indicate that only Type A was generated. XRPD comparison results are shown in FIG. 15 and FIG. 16. The DSC curves in FIG. 17 shows only a sharp endotherm at about 95°

TABLE 12

| Experiment ID | Solvent (v/v) | Result |
|---|---|---|
| 810055-04-A1^{#} | IPA | Type A |
| 810055-04-A2^{#} | 2-MeTHF | Type A |
| 810055-04-A3 | n-Heptane | Type A |
| 810055-04-A5 | DMAc/H₂O (1:9) | Type A |
| 810055-04-A6* | THF/n-heptane (1:1) | Type A |
| 810055-04-A7* | MTBE/n-heptane (1:1) | Type A |

*Clear solution was obtained after slurry at 50° C. for 3 days, which was transferred to slurry at 5° C. for another 2 days.
^{#}Clear solution was obtained after slurry at 50° C. for 3 days and then at 5° C. for 2 days, which was transferred to evaporate at RT.

Additional slurry conversion experiments were also conducted at 50° C. in different solvent systems as shown in Table 13. About 25˜50 mg of a starting material (817506-01-A) was suspended in 0.4 mL of a solvent in an HPLC vial. After the suspension was magnetically stirred for about 4 days at 50° C., the remaining solids were isolated for XRPD analysis. Results summarized in Table 13 indicate that Type A, a mixture of Type A+Impurity B, and amorphous sample were generated using the different solvent systems.

TABLE 13

| Experiment ID | Solvent (v/v) | Result |
|---|---|---|
| 817506-07-A1^{&} | IPA | Type A + Impurity B |
| 817506-07-A2^{#} | MIBK | Type A |
| 817506-07-A3^{#} | 2-MeTHF | Type A + Impurity B |
| 817506-07-A4^{&} | 1,4-Dioxane | Type A |
| 817506-07-A5 | n-Heptane | Type A + Impurity B |
| 817506-07-A6 | H₂O | Type A |
| 817506-07-A7** | n-Butanol/toluene (1:1) | Amorphous (2 peaks) |
| 817506-07-A8* | CHCl₃/cyclohexane (1:2) | Type A |
| 817506-07-A9 | DMAc/H₂O (1:9) | Type A + Impurity B |
| 817506-07-A10^{&} | Acetone/n-heptane (1:1) | Type A |
| 817506-07-A11 | ACN/H₂O (1:9) | Type A |
| 817506-07-A12^{&} | THF/n-heptane (1:1) | Type A + Impurity B |
| 817506-07-A13^{&} | EtOH/1,4-dioxane (1:1) | Type A |
| 817506-07-A14* | MTBE/n-heptane (1:1) | Type A + Impurity B |

*Clear solutions were obtained after slurry at 50° C. for 4 days, which were transferred to slurry between 50° C. and 5° C. (3 cycles).
^{#}Clear solutions were obtained after slurry at RT for 3 days followed by between 50° C. and 5° C. (3 cycles), which were transferred to slurry at −20° C. to obtain solids.
^{&}Clear solutions were obtained after slurry at RT for 3 days and between 50° C. and 5° C. (3 cycles), followed by slurry at −20° C. Add anti-solvent H₂O or n-heptane and slurry at RT for 3 days.
**Clear solution was obtained after adding anti-solvent n-heptane, which was transferred to evaporate at RT.

About 20 mg of starling material (8152100-01-A) was suspended in 0.5 mL of corresponding solvent in an HPLC vial. After the suspensions were magnetically stirred (˜1000 rpm) at −20° C. for ˜7 days, the remaining solids were isolated by centrifugation for XRPD analysis. Results summarized in Table 14 showed that Type A was generated.

TABLE 14

| Experiment ID | Solvent (v/v) | Result |
|---|---|---|
| 8152100-06-A1 | m-Xylene | Type A |
| 8152100-06-A2 | EtOH/n-Heptane (1:4) | Type A* |
| 8152100-06-A3 | MEK/n-Heptane (1:4) | Type A* |
| 8152100-06-A4 | Ethyl formate/n-Hexane (1:4) | Type A |
| 81$2100-06-A5 | Toluene/Cyclohexane (1:4) | Type A |
| 8152100-06-A6 | n-Butanol/Cyclohexane (1:4) | Type A* |
| 8152100-06-A7 | EtOAc/n-Hexane (1:4) | Type A |
| 8152100-06-A8 | Toluene/n-Heptane (1:4) | Type A |
| 8152100-06-A9 | 2,2,2-Trifluoroethanol/n-Hexane (1:4) | Type A* |
| 8152100-06-A10 | IPA/Cyclohexane (1:4) | Type A* |
| 8152100-06-A11 | 2-MeTHF/n-Heptane (1:4) | Type A |
| 8152100-06-A12 | Pyridine/n-Heptane (1:4) | Type A* |
| 8152100-06-A13 | m-Xylene/n-Hexane (1:4) | Type A |
| 8152100-06-A14 | Isobutanol/n-Heptane (1:4) | Type A* |
| 8152100-06-A15 | Cumene/n-Heptane (1:4) | Type A |

*Clear solution was obtained after slurry at −20° C., which was transferred to evaporation at RT.

About 20 mg of starting material (8152100-01-A) was suspended in 0.5 mL of corresponding solvent in an HPLC vial. The suspensions were then heated to 50° C. and equilibrated at 50° C. for 2 hrs. Set the temperature dropping from 50° C. to 5° C. over 450 min and keep at 5° C. for 2 hrs. Raise temperature to 50° C. over 30 min. Repeat the step of heating and cooling twice and then cool to 5° C. over 450 min. The obtained solids were kept isothermal at 5° C. before isolation for XRPD analysis. Results summarized in Table 15 showed that Type A was obtained.

TABLE 15

| Experiment ID | Solvent (v/v) | Result |
|---|---|---|
| 8152100-07-A1 | H₂O | Type A |
| 8152100-07-A2 | MTBE | Type A* |
| 8152100-07-A3 | Ethyl lactate/n-Heptane (1:9) | Type A |
| 8152100-07-A4 | Chlorobenze/n-Heptane (1:9) | Type A |
| 8152100-07-A5 | MEK/n-Hexane (1:9) | Type A^{#} |
| 8152100-07-A6 | CPME/n-Hexane (1:9) | Type A |
| 8152100-07-A7 | Pyridine/n-Hexane (1:9) | Type A* |
| 8152100-07-A8 | 1,2-Dimethoxyethane/n-Hexane (1:9) | Type A |
| 8152100-07-A9 | Anisole/Cyclohexane (1:9) | Type A |
| 8152100-07-A10 | Isoamyl alcohol/Cyclohexane (1:9) | Type A* |
| 8152100-07-A11 | Acetone/Cyclohexane (1:9) | Type A* |
| 8152100-07-A12 | EtOAc/H₂O (1:9) | Type A |
| 8152100-07-A13 | MeOH/H₂O (1:9) | Type A |
| 8152100-07-A14 | DMSO/H₂O (1:9) | Type A |
| 8152100-07-A15 | ACN/H₂O (1:9) | Type A |

*Clear solution was obtained after slurry with temperature cycling and slurry at −20° C., which was transferred to evaporation at RT.
^{#}Clear solution was obtained after slurry with temperature cycling. Solid was obtained after slurry at −20° C.

### Example 6: Preparation of Crystalline Type A of N-(3,5-difluorobenzyl)-N-hydroxy-2,2-dimethylbutanamide by Slow Evaporation at RT and at 20° C., and by Fast Evaporation

Slow evaporation experiments were performed under six conditions. About 15 mg of a starting material (810055-01-A or 810048-48-B) was dissolved in 2.0 mL of a solvent as shown in Table 16 in a 3-mL glass vial. If the material was not dissolved completely, suspensions were filtered using a 0.45 μm PTFE membrane and the filtrates were used for the follow-up steps. The visually clear solutions were subjected to evaporation at RT in vials sealed by Parafilm® (poke 5 small holes). The solids were isolated for XRPD analysis, and the results summarized in Table 16 indicated that only Type A was observed. XRPD and DSC results are shown in FIG. 18 and FIG. 19.

TABLE 16

| Experiment ID | Solvent | Result |
|---|---|---|
| 810055-05-A1 | MTBE | Type A |
| 810053-05-A2 | DCM | Type A |
| 810048-19-B3 | Toluene | Type A |

Additional low evaporation experiments were performed under ten conditions. About 15 mg of a starting material (817506-01-A) was dissolved in 2.0 mL of a solvent as shown in Table 17 in a 3-mL glass vial. If the material was not dissolved completely, suspensions were filtered using a 0.45 μm PTFE membrane and the filtrates were used for the follow-up steps. The visually clear solutions were subjected to evaporation at RT in vials sealed by Parafilm® (poke 4 small holes). The solids were isolated for XRPD analysis, and the results summarized in Table 17 indicated that Type A and a mixture of Type A+Impurity B were observed using the different solvent systems.

TABLE 17

| Experiment ID | Solvent (v/v) | Result |
|---|---|---|
| 817506-08-A1 | MeOH | Type A |
| 817506-08-A2 | EtOH | Type A |
| 817506-08-A3 | Acetone | Type A |
| 817506-08-A4 | BtOAc | Type A |
| 817506-08-A5 | THF | Type A |
| 817506-08-A6 | MTBE | Type A + Impurity B |
| 817506-08-A7 | DCM | Type A + Impurity B |
| 817506-08-A8 | CHCl₃ | Type A |
| 817506-08-A9 | ACN | Type A |
| 817506-08-A10 | Toluene | Type A + Impurity B |

Around 20 mg of starting material (8152100-01-A) was dissolved in 2.0 mL corresponding solvent in a 3-mL glass vial and oscillation and sonication was performed to dissolve solid. The sample was filtered using PTFE membrane (pore size 0.45 μm). The clear solutions were subjected to evaporation at RT in vials sealed by Parafilm® (poke 4 small holes). The solids were isolated for XRPD analysis, and the results summarized in Table 18 showed that Type A was observed.

TABLE 18

| Experiment ID | Solvent (v/v) | Result |
|---|---|---|
| 8152100-08-A1 | IPA | Type A* |
| 8152100-08-A2 | MIBK | Type A* |
| 8152100-08-A3 | MEK | Type A |
| 8152100-08-A4 | IPAc | Type A |
| 8152100-08-A5 | Ethyl formate | Type A |
| 8152100-08-A6 | 2,2,2-Trifluoroethanol | Type A* |
| 8152100-08-A7 | 2-MeTHF | Type A |
| 8152100-08-A8 | CH₂Br₂ | Type A* |
| 8152100-08-A9 | Ether | Type A |
| 8152100-08-A10 | CPME | Type A* |
| 8152100-08-A11 | MeOH/DCM (1:1) | Type A |
| 8152100-08-A12 | THE/n-Heptane (4:1) | Type A |

*No solid was obtained after evaporation at RT for 2 weeks. The sample was transferred to dry at RT under vacuum.

Around 20 mg of starting material (8152100-01-A) was dissolved in 0.5-2.0 mL corresponding solvent in a 3-mL glass vial and oscillation and sonication was performed to dissolve solid. The sample was filtered using PTFE membrane (pore size 0.45 μm). The clear solutions were subjected to evaporation at −20° C. in vials sealed by Parafilm® (poke 4 small holes). The solids were isolated for XRPD analysis, and the results summarized in Table 19 showed that Type A was observed.

TABLE 19

| Experiment ID | Solvent (v/v) | Result |
|---|---|---|
| 8152100-23-A1 | EtOH | Type A* |
| 8152100-23-A2 | Acetone | Type A* |
| 8152100-23-A3 | EtOAc | Type A* |
| 8152100-23-A4 | MTBE | Type A* |
| 8152100-23-A5 | DCM | Type A |
| 8152100-23-A6 | CHCl₃ | Type A |
| 8152100-23-A7 | Ethyl formate | Type A* |
| 8152100-23-A8 | ACN | Type A* |

*Clear solution was obtained after evaporation at −20° C. for 17 days and transferred to evaporation at RT.

Around 20 mg of starting material (8152100-01-A) was dissolved in 0.2-2.0 mL corresponding solvent in a 3-mL glass vial and oscillation and sonication was performed to dissolve solid. The sample was filtered using PTFE membrane (pore size 0.45 μm). The clear solutions were subjected to evaporation at 80° C., and the results summarized in Table 20 showed that Type A was observed.

TABLE 20

| Experiment ID | Solvent (v/v) | Result |
|---|---|---|
| 8152100-09-A1 | MeOH | Type A |
| 8152100-09-A2 | Isobutanol | Type A |
| 8152100-09-A3 | Acetone | Type A |
| 8152100-09-A4 | 1,4-Dioxane | Type A |
| 8152100-09-A5 | THF | Type A |
| 8152100-09-A6 | Methyl acetate | Type A |
| 8152100-09-A7 | n-Butyl acetate | Type A |
| 8152100-09-A8 | ACN | Type A |
| 8152100-09-A9 | m-Xylene | Type A* |
| 8152100-09-A10 | CHCl₃/n-Heptane (4:1) | Type A |
| 8152100-09-A11 | EtOAc/Cyclohexane (4:1) | Type A |
| 8152100-09-A12 | EtOH/H₂O (4:1) | Type A |

*No solid was obtained after evaporation at 80° C. for 3 days. The sample was transferred to dry at RT under vacuum.

### Example 7: Preparation of Crystalline Type A of N-(3,5-difluorobenzyl)-N-hydroxy-2,2-dimethylbutanamide by Slow Cooling and Fast Cooling

Slow cooling experiments were conducted in three solvent systems. About 20 mg of a starting material (810055-01-A or 810048-18-B) was suspended in 0.1˜0.2 mL of a solvent as shown in Table 21 in a 3-mL glass vial at RT. The suspension was then heated to 50° C., equilibrated for about two hours, and the suspension was filtered into a new vial using a 0.45 μm PTFE membrane. The solution or filtrate was slowly cooled down to 5° C. at a rate of 0.1° C./min. The obtained solids were kept isothermal at 5° C. before isolation for XRPD analysis. Results summarized in Table 21 indicated only Type A was obtained. XRPD and DSC results are displayed in FIG. 20 and FIG. 21.

TABLE 21

| Experiment ID | Solvent (v/v) | Result |
|---|---|---|
| 810055-06-A2* | MIBK/2-MeTHF (1:1) | Type A |
| 810055-06-A3* | n-BuOH/n-heptane (1:1) | Type A |
| 810048-19-B5^{#} | Acetone/n-heptane (1:2) | Type A |

*Clear solution was obtained after slow cooling and further stand at −20° C., which was transferred to

Additional slow cooling experiments were conducted in eight solvent systems as shown in Table 22. About 20 mg of a starting material (817506-01-A) was suspended in 0.1˜2.0 mL of corresponding solvent in a 3-mL glass vial at RT. The suspension was then heated to 50° C., equilibrated for about two hours, and filtered into a new vial using a 0.45 μm PTFE membrane. The filtrates were slowly cooled down to 5° C. at a rate of 0.1° C./min. The obtained solids were kept isothermal at 5° C. before isolation for XRPD analysis. Results summarized in Table 22 indicated Type A and a mixture of Type A+Impurity B were obtained using the different solvent systems.

TABLE 22

| Experiment ID | Solvent (v/v) | Result |
|---|---|---|
| 817506-09-A1* | EtOH/n-heptane (1:2) | Type A |
| 817506-09-A2 | Acetone/n-heptane (1:2) | Type A + Impurity B |
| 817506-09-A3* | EtOAc/n-heptane (1:2) | Type A |
| 817506-09-A4* | MTBE | Type A |
| 817506-09-A6 | CHCl₃/n-heptane (1:2) | Type A |
| 817506-09-A7 | NMP/H₂O (1:4) | Type A |
| 817506-09-A8^{#} | MIBK/2-MeTHF (1:1) | Type A + Impurity B |
| 817506-09-A9^{#} | n-BnOH/n-heptane (1:2) | Type A + Impurity B |

*Clear solution was obtained after slow cooling, which were transferred to stand at −20° C.
^{#}Clear solution was obtained after slow cooling an further stand at −20° C. Transferred to evaporate at RT.

About 20 mg of starting material (8152100-01-A) was suspended in 0.2-1.0 mL of corresponding solvent in a 3-mL glass vial. The suspension was then heated to 50° C. and equilibrated at 50° C. for 2 hrs before being filtered to a new vial through PTFE membrane (pore size 0.45 μm). Filtrates were slowly cooled down to 5° C. at a rate of 0.1° C./min. The obtained solids were kept isothermal at 5° C. before isolation for XRPD analysis. Results in Table 23 indicated that Type A was generated.

TABLE 23

| Experiment ID | Solvent (v/v) | Result |
|---|---|---|
| 8152100-10-A1 | Cumene | Type A* |
| 8152100-10-A2 | IPAc/n-Hexane (1:4) | Type A |
| 8152100-10-A3 | Pyridine/n-Hexane (1:9) | Type A^{#} |
| 8152100-10-A4 | MeOH/H₂O (1:4) | Type A** |
| 8152100-10-A5 | ACN/H₂O (1:4) | Type A\hat{ } |
| 8152100-10-A6 | MEK/n-Heptane (1:4) | Type A^{#} |
| 8152100-10-A7 | Ethyl lactate/n-Heptane (1:4) | Type A^{#} |
| 8152100-10-A8 | Anisole/Cyclohexane (1:4) | Type A* |

*Clear solution was obtained after slow cooling to 5° C. and slurry at −20° C. So the sample was transferred to evaporation at RT.
**Clear solution was obtained after slow cooling to 5° C., slurry at −20° C. and evaporation at RT. So the sample was transferred to dry at RT under vacuum.
^{#}Clear solution was obtained after slow cooling to 5° C. So the sample was transferred to evaporation at RT.
\hat{ }Clear solution was obtained after slow cooling to 5° C., slurry at −20° C. and evaporation at RT. So the sample was transferred to dry at RT under vacuum. Compared with the pattern of Type A, an extra weak diffraction peak was observed. As the amount of the sample was very limited, the sample was insufficient for XRPD re-test or further investigation.

About 20 mg of starting material (8152100-01-A) was suspended in 0.3-0.5 mL of corresponding solvent in a 3-mL glass vial. The suspension was then heated to 50° C. and equilibrated at 50° C. for 2 hrs before being filtered to a new vial through PTFE membrane (pore size 0.45 μm). Filtrates were placed at −20° C. for precipitation. The obtained solids were isolated for XRPD analysis. Results in Table 24 indicated that Type A was generated.

TABLE 24

| Experiment ID | Solvent (v/v) | Result |
|---|---|---|
| 8152100-11-A1 | n-Butanol/n-Hexane (1:4) | Type A |
| 8152100-11-A2 | MIBK/n-Hexane (1:4) | Type A |
| 8152100-11-A3 | IPAc/Cyclohexane (1:4) | Type A |
| 8152100-11-A4 | MTBE/Cyclohexane (1:4) | Type A |
| 8152100-11-A5 | Benzyl alcohol/Cyclohexane (1:4) | Type A* |
| 8152100-11-A6 | Methy acetate/n-Heptane (1:4) | Type A |
| 8152100-11-A7 | Toluene/n-Heptane (1:4) | Type A |
| 8152100-11-A8 | DCM/n-Heptane (1:4) | Type A |

*Sample was frozen at −20° C. and turned clear at RT. The sample was transferred to evaporation at RT.

### Example 8: Preparation of Crystalline Type A or Type C of N-(3,5-difluorobenzyl)-N-hydroxy-2,2-dimethylbutanamide by Liquid Vapor Diffusion

Four liquid vapor diffusion experiments were conducted. Approximately 15 mg of a starting material (810055-01-A or 810048-18-B) was dissolved in 0.1˜0.3 mL of a solvent as shown in Table 25 in a 3-mL glass vial to obtain a clear solution. The 3-mL vial was then placed into a 20-mL vial with 4 mL of volatile solvents (as shown in the “anti-solvent” column in Table 25). The 20-mL vial was sealed with a cap and kept at RT allowing sufficient time for the organic vapor to interact with the solution. The precipitates were isolated for XRPD analysis. After 7˜10 days of diffusion, clear solutions were transferred for evaporation at RT, and the obtained solids were tested by XRPD. The results were summarized in Table 25 and XRPD patterns are shown in FIG. 22; only Type A was generated. DSC curves in FIG. 23 showed only a sharp endotherm at about 95° C. (onset).

TABLE 25

| Experiment ID | Solvent | Anti-solvent | Result |
|---|---|---|---|
| 810055-07-A1* | MIBK | 2-MeTHF | Type A |
| 810055-07-A2 | Acetone | Cyclohexane | Type A |
| 810055-07-A3* | EtOAc | Cyclohexane | Type A |
| 810048-19-B6 | DMSO | H₂O | Type A |

*Clear solution was obtained after 7 days diffusion and then transferred to evaporate at RT.

Additional ten liquid vapor diffusion experiments were conducted. Approximately 15 mg of a starting material (817506-01-A) was dissolved in 0.1˜0.5 mL of a solvent as shown in Table 26 in a 3-mL glass vial to obtain a clear solution. The 3-mL vial was then placed into a 20-mL vial with 4 mL of volatile solvents (as shown in the “anti-solvent” column in Table 26). The 20-mL vial was sealed with a cap and kept at RT allowing sufficient time for the organic vapor to interact with the solution. The precipitates were isolated for XRPD analysis. After seven days of diffusion, clear solutions were transferred for evaporation at RT, and the obtained solids were tested by XRPD. The results summarized in Table 26 showed that Type A, Type C, and a mixture of Type A+Impurity B were observed using the different solvent systems.

TABLE 26

| Experiment ID | Solvent | Anti-solvent | Result |
|---|---|---|---|
| 817506-10-A1* | EtOH | n-Heptane | Type A |
| 817506-10-A2* | THF | | Type A |
| 817506-10-A3* | IPAc | | Type A |
| 817506-10-A4* | MeOH | 2-MeTHF | Type A |
| 817506-10-A5* | MIBK | | Type A + Impurity B |
| 817506-10-A6* | ACN | | Type A |
| 817506-10-A7 | Acetone | Cyclohexane | Type A + Impurity B |
| 817506-10-A8 | EtOAc | | Type A + Impurity B |
| 817506-10-A9 | 1,4-Dioxane | H₂O | Type A |
| 817506-10-A10 | DMSO | | Type C |

*Clear solution was obtained after 7 days diffusion and then transferred to evaporate at RT.

### Example 9: Preparation of Crystalline Type A of N-(3,5-difluorobenzyl)-N-hydroxy-2,2-dimethylbutanamide by Grinding

Three grinding experiments were conducted. Approximately 20 mg of a starting material (810055-01-A) was manually ground in an agate mortar using a pestle for about 5 min. The solids were checked by XRPD (shown in FIG. 24) and the results summarized in Table 27 showed that only Type A was obtained. DSC curves in FIG. 27 showed only a sharp endotherm at about 95° C. (onset).

TABLE 27

| Experiment ID | Solvent | Result |
|---|---|---|
| 810055-08-A1 | N/A | Type A |
| 810055-08-A2 | EtOH | Type A |
| 810055-08-A3 | MTBE | Type A |

Additional four grinding experiments were conducted. Approximately 25 mg of a starting material (817506-01-A) was manually ground in an agate mortar using a pestle for about 5 min. The solids were checked by XRPD and the results summarized in Table 28 showed that only Type A and a mixture of Type A+Impurity B was obtained using the different solvent systems.

TABLE 28

| Experiment ID | Solvent | Result |
|---|---|---|
| 817506-12-A2 | H₂O | Type A |
| 817506-12-A3 | EtOH | Type A + Impurity B |
| 817506-12-A4 | MTBE | Type A + Impurity B |

### Example 10: Preparation of Crystalline Type A or Amorphous Form of N-(3,5-difluorobenzyl)-N-hydroxy-2,2-dimethylbutanamide by a Polymer-Induced Method, a Polymer/Ionic Liquid Induced Crystallization Method and a Co-Crystal Screening Method

Polymer-induced experiments were performed with two sets of polymer mixtures in six solvents as shown in Table 29. Approximately 15 mg of a starting material (817506-01-A) was dissolved in 2.0 mL of a solvent to obtain a clear solution in a 3-mL vial. About 2 mg of the polymer mixture was added into the 3-mL glass vial. The solutions were subjected to evaporation at RT with vials sealed by Parafilm® (poke 3˜5 small holes). The solids were isolated for XRPD analysis. Results summarized in Table 29 showed that Type A and an amorphous sample were obtained.

TABLE 29

| Experiment ID | Solvent (v/v) | Polymer | Result |
|---|---|---|---|
| 817506-11-A1 | IPA | Polymer | Type A (3 peaks) |
| 817506-11-A2 | MIBK | mixture A | Type A |
| 817506-11-A3 | ACN/H₂O (1:1) | | Type A |
| 817506-11-A4 | IPAc | Polymer | Amorphous |
| 817306-11-A5 | THF | mixture B | Type A |
| 817506-11-A6 | MeOH/DCM (1:1) | | Type A |

*: 0.1 mL solvent was added and filtration was not performed.
Polymer mixture A: polyvinyl pyrrolidone (PVP), polyvinyl alcohol (PVA), polyvinylchloride (PVC), polyvinyl acetate (PVAC), hypromellose (HPMC), methyl cellulose (MC) (mass ratio of 1:1:1:1:1:1)
Polymer mixture B: polycaprolactone (PCL), polyethylene glycol (PEG), poly(methyl methacrylate) (PMMA) sodium alginate (SA), and hydroxyethyl cellulose (HEC) (mass ratio of 1:1:1:1:1).

Eight experiments were set up in different solvents by polymer/ionic liquid induced crystallization. Around 20 mg of starting material (815298-01-A) was dissolved in corresponding solvent in an HPLC vial and oscillation and sonication was performed to dissolve solid. The sample was filtered using PTFE membrane (pore size 0.45 μm) to prepare saturated clear solution. Polymer or ionic liquid was then added into the solutions and the solutions were magnetically stirred (˜1000 rpm) at RT. The obtained solids were isolated for XRPD analysis. The results summarized in Table 30 showed that only Type A was observed.

TABLE 30

| Experiment | | | Re- |
|---|---|---|---|
| ID | Solvent (v/v) | Polymer/ionic liquid | sult |
| 8152100-24-A1 | n-Heptane/ | Polymer mixture A | Type A |
| | IPAc (1:1) | | |
| 8152100-24-A2 | MeOH/H₂O | | Type A |
| | (1:1) | | |
| 8152100-24-A3 | m-Xylene | Polymer mixtureB | Type A |
| 8152100-24-A4 | Acetone/ | | Type A |
| | n-Heptane | | |
| | (1:1) | | |
| 8152100-25-A1 | Toluene | 1-ethyl-3-methylimidazolium | Type A |
| | | trifluoroacetate | |
| 8152100-25-A2 | ACN/H₂O | 1-butyl-3-methylimidazolium | Type A |
| | (2:1) | tetrafluoroborate | |
| 8152100-23-A3 | MTBE/n- | 1-butyl-3-methylimidazolium | Type A |
| | Heptane (1:1) | hexafluorophosphate | |
| 8152100-25-A4 | CHCl₃/ | 1,3-dimethylimidazolium | Type A |
| | n-Heptane (1:1) | dimethylphosphate | |

Polymer mixture A: polyvinyl pyrrolidone (PVP), polyvinyl alcohol (PVA), polyvinylchloride (PVC), polyvinyl acetate (PVAC), hypromellose (HPMC), methyl cellulose (MC) (mass ratio of 1:1:1:1:1:1)
Polymer mixture B: polycaprolactone (PCL), polyethylene glycol (PEG), poly (methyl methacrylate) (PMMA) sodium alginate (SA), and hydroxyethyl cellulose (HEC) (mass ratio of 1:1:1:1:1).

Starting from Type A (8152100-01-A), a total of 68 co-crystal screening each HPLC vial with a desired molar ratio of 1:1. Then, 0.5 mL of corresponding solvent (Acetone, EtOAc, MTBE) was added to the vials and all the samples were magnetically stirred at RT for about six days. Solids were isolated for XRPD analysis. Clear solutions were transferred to slurry at 5° C. If clear solution was still obtained, evaporation at RT or anti-solvent addition was performed to induce precipitation). For experiments using EtOH as solvent, about 20 mg of starting material (8152100-01-A) and equimolar corresponding co-former were transferred to an agate mortar. After manual grinding for ˜3 min, solids were collected for XRPD analysis. As summarized in Table 31, only Type A, co-former or mixtures of Type A and co-former were obtained.

TABLE 31

| | | Solvent | | | |
|---|---|---|---|---|---|
| # | Co-former | A: EtOH^{[1]} | B: Acetone | C: EtOAc | D: MTBE |
| 0 | Blank | Not performed | Type A^{[4]} | Type A^{[4]} | Type A^{[4]} |
| 1 | Maleic acid | Type A + | Type: A + co- | Type A + co- | Type A +co- |
| | | co-former | former^{[3]} | former^{[3]} | former^{[3]} |
| 2 | Fumaric acid | Type A + co- | Type A + co- | Type A + co- | Type A + Co- |
| | | former | former | former | former |
| 3 | Succinic acid | Type A + co- | Type A + co- | Type A + co- | Type A + co- |
| | | former | former^{[3]} | former | former |
| 4 | Citric acid | Type A + co- | Type A + co- | Type A + co- | Co-former |
| | | former | former^{[4]} | former | |
| 5 | Malic acid | Type A + co- | Type A^{[4]} | Co-former^{[3]} | Type A + co- |
| | | former | | | form^{[3]} |
| 6 | Tartaric acid | Type A + co- | Type A + co- | Type A + co- | Type A + co- |
| | | former | former^{[3]} | former | former |
| 7 | Gentisic acid | Type A + co- | Type A + co- | Type A + co- | Type: A + co- |
| | | former | former^{[3]} | former^{[3]} | former^{[3]} |
| 8 | Benzoic acid | Type A + co- | Type A^{[3]} | Type A + co- | Type A + co- |
| | | former | | former^{[4]} | former^{[4]} |
| 9 | Nicotinic acid | Type A | Type A^{[4]} | Type A^{[4]} | Type A^{[4]} |
| 10 | Benzenesulfonic | Type A + peak | Type A + peak^{[4]} | Different | Different |
| | acid | | | XRPD^{[7]} | XRPD^{[7]} |
| 11 | Naphthalene- | Type A + co- | Type A + co- | Co-former | Co-former |
| | 1,5-disulfonic | former | former^{[4]} | | |
| | acid | | | | |
| 12 | Arginine | Type A + co- | Co-former ^{[6]} | Type A + co- | Type A + co- |
| | | former | | former | former |
| 13 | Lysine | Type A | Co-former ^{[5]} | Type A + co- | Type A + co- |
| | | | | former | former |
| 14 | Choline | Type A | Type A^{[4]} | Type A^{[4]} | Type A^{[4]} |
| 15 | Trometamol | Type A + co- | Type A + co- | Co-former^{[2]} | Type A + co- |
| | | former | toner | | former |
| 16 | Piperazine | Type A | Type A^{[4]} | Type A^{[4]} | Type A + peak^{[4]} |
| 17 | Ethanolamine | Type A | Type A^{[4]} | Type A^{[4]} | Type A^{[4]} |

^{[1]}Experiments were performed via grinding.
^{[2]}Clear solution was obtained after slurry at RT for 6 days. Solid was obtained after slurry at 5° C. for 10 days.
^{[3]}Clear solution was obtained after slurry at RT for 6 days and slurry at 5° C. for 10 days. Solid was obtained after anti-solvent addition (n-Heptane).
^{[4]}Clear solution was obtained after slurry at RT for 6 days and at 5° C. for 10 days. No solid was obtained after adding anti-solvent (n-Heptane). The solution was transferred to evaporation at RT.
^{[5]} Based on the results of re-preparation and control experiments, the sample with different XRPD pattern was speculated to be a new form of lysine (refer to Appendix 7.1.2).
^{[6]} Based on the results of re-preparation and controlled experiments, the sample with different XRPD pattern was speculated to be a new form of arginine (refer to Appendix 7.1.1).
^{[7]}Clear solution was obtained after slurry at RT for 6 days. Limited amount of solid was obtained after slurry at 5° C. for 10 days. XRPD pattern of the obtained solid was different from Type A and benzenesulfonic acid.
¹H NMR result showed that several peaks that could not be attributed to API or benzenesulfonic acid were observed, indicating degradation of the sample. Re-preparation was performed in MTBE and only Type A was obtained.

### Example 11: Preparation of Crystalline Type A of N-(3,5-difluorobenzyl)-N-hydroxy-2,2-dimethylbutanamide Using Type A Sample

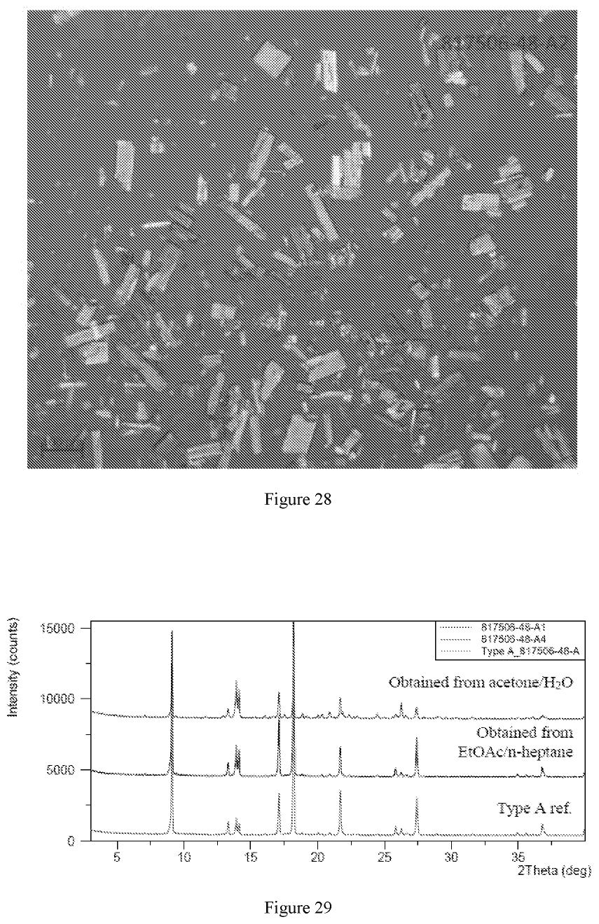

Using a Type A sample (817506-48-A) as the starting material, five experiments were performed to prepare Type A at 200 mg scale, and the results are summarized in Table 32. Detailed procedures are as follows.
1. Dissolve about 200 mg of Type A sample (817506-48-A) by a solvent to obtain clear solutions;
2. Add an anti-solvent dropwise into the solutions when stirring;
3. Isolate the obtained suspensions by centrifugation (10000 rpm, 2 min) for XRPD analysis.
4. Dry the solids at RT under vacuum for about 16 hrs.
   As shown in Table 32, a sample (817506-48-A2) was obtained from MeOH/H2O using Type A as a starting material, and TGA, DSC, and PLM characterization were also performed. As shown in FIG. 27, a weight loss of about 0.8% at up to 100° C. and a sharp endotherm at 94.8° C. (onset) were observed on TGA/DSC curves. Based on XRPD (FIGS. 26 and 29) and DSC results, the batch of the sample (817506 48-A2) is pure Type A. PLM image in FIG. 28 showed Type A (817506-48-A2) comprises blade-like crystals.

TABLE 32

| Exp. ID | 817506-48-A1 | 817506-48-A2 | 847506-48-A3 | 817506-48-A4 | 817506-48-A5 |
|---|---|---|---|---|---|
| Starting form | Type A | Type A | Type A | Type A | Type A |
| Weight (mg) | 191.5 | 192.4 | 193.9 | 191.5 | 189.0 |
| Solvent | Acetone | MeOH | THF | EtOAc | Acetone |
| Vol. of solvent | 1.0 | 10 | 2.0 | 1.0 | 1.0 |
| (mL) | | | | | |
| Anti-solvent | H₂O | H₂O | H₂O | n-Heptane | Cyclohexane |
| Vol. of anti- | 3.0 | 1.0 | 4.0 | 9.0 | 15.0 |
| solvent (mL) | | | | | |
| Final form | Type A* | Type A | Oil | Type A | Clear |
| Weight of | 172.3 | 152.3 | N/A | 34.0 | N/A |
| product (mg) | | | | | |
| Yield (%) | 90.0 | 79.2 | N/A | 17.8 | N/A |

*Oil-like sample was obtained and converted to Type A after slurry.

### Example 12: Preparation of Crystalline Type A of N-(3,5-difluorobenzyl)-N-hydroxy-2,2-dimethylbutanamide Using Type A+Impurity B as Starting Material

In order to investigate if pure Type A could be obtained from a mixture of Type A+Impurity B, another batch of experiment was performed in a MeOH/H2O system using an A+B sample (817506-38-A) as the starting material. Detailed procedures are as follows.
1. Weigh 100.4 mg of an A+B sample (817506-38-A, Lot No.: SN-023-91-17) into a 5-mL glass vial. Dissolve the solids in 0.5 mL of MeOH to obtain a clear solution.
2. Add 0.25 mL of anti-solvent H₂O dropwise into the clear solution when stirring. a suspension was obtained.
3. Isolate the solids by centrifugation (10000 rpm, 2 min) for XRPD analysis.
4. Dry the solids at RT under vacuum for about 16 hrs. A total of 71.3 mg of solids were obtained.
   XRPD pattern in FIG. 30 showed the solids obtained (810048-03-A1) was Type A without diffraction peaks of Impurity B. DSC curve in FIG. 31 showed only a sharp endotherm at 94.3° C. (onset). Based on the data, pure Type A was obtained.

### Example 13: Evaluation of Type A—Equilibrium Solubility in Water

Equilibrium solubility of Type A in H₂O was measured at RT. Specifically, 10.0 mg of a Type A sample (817506-48-A2) was suspended into 1 mL of H₂O (˜1000 rpm) at RT. After 24 hrs, the suspension was centrifuged (10000 rpm, 5 min, RT) followed by filtration (0.45 μm PTFE membrane). The supernatant (first few drops were discarded) was analyzed for HPLC solubility and pH, and the residual solids were used for XRPD analysis. Results were summarized in Table 33. The measured solubility of Type A was 0.15 mg/mL. As shown in FIG. 32, no form change was observed for Type A after suspending in H₂O at RT for about 24 hrs

TABLE 33

| Crystal form | Sample ID | Solubility (mg/mL) | pH | Form change |
|---|---|---|---|---|
| Type A | 817506-48-A2 | 0.15 | 8.7 | No |

### Example 14: Evaluation of Type A—Solid State Stability

To evaluate the solid state stability, a Type A sample (817506-48-A2) was stored under 80° C. for one day, and 25° C./60% RH and 40° C./75% RH for one week each. All the samples were characterized using XRPD, DSC, and HPLC, with the results summarized in Table 34. No form change or decrease in purity was observed for the Type A sample (817506-48-A2) under all the conditions, indicating Type A possessed good solid state stability. HPLC chromatogram overlay is shown in FIG. 33. XRPD results were displayed in FIG. 34. DSC characterization results were shown from FIG. 35 to FIG. 37. Only one endotherm at about 95° C. was observed, indicating the solids under all the conditions were pure Type A.

TABLE 34

| | Purity of | 80° C./1 d | | 25° C./60% RH/1 w | | 40° C./75% RH/1 w | |
|---|---|---|---|---|---|---|---|
| Initial Form | Initial | % of | Final | % of | Final | % of | Final |
| (Sample ID) | (area %) | Initial | Form | Initial | Form | Initial | Form |
| Type A | 100.0 | 100.0 | Type A | 100.0 | Type A | 100.0 | Type A |
| (817506-48-A2) | | | | | | | |

### Example 15: Evaluation of Type A—Photostability

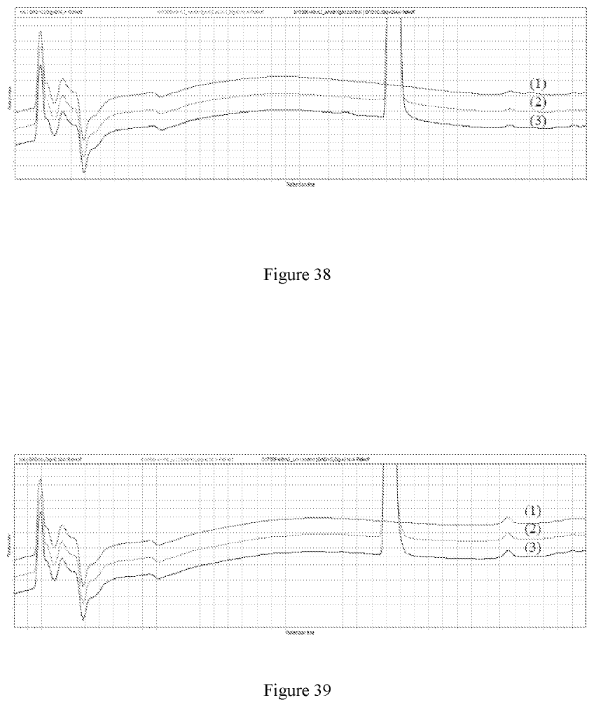

Photo stability of Type A (817506-48-A2) was assessed under both white light (1,200,000 Lux-hrs) and ultraviolet (200 W·hrs/m²) conditions according to the ICH guideline. The results were summarized in Table 35. As HPLC chromatogram in FIG. 38 and FIG. 39, no purity decrease was observed for Type A after photo stability evaluation. XRPD results are displayed from FIG. 40 to FIG. 41. Only an endotherm at about 95° C. was observed on the DSC curves (FIGS. 42-43). Combined with XRPD results, no form change was detected after photo stability evaluation under both white light and ultraviolet, indicating Type A possesses good photo stability.

TABLE 35

| Sample | Form | Purity (area %) |
|---|---|---|
| Initial | Type A | 100.0 |
| White light | Type A | 100.0 |
| White light_Control* | Type A | 100.0 |
| UV | Type A | 100.0 |
| UV_Control* | Type A | 100.0 |

*Sample in a foil-covered vial under both conditions is performed as controlled trials.

### Example 16: Evaluation of Type A—Hygroscopicity

To investigate the solid from stability as a function of humidity, a DVS isotherm plot of Type A (817506-48-A2) was collected at 25° C. between 0 and 95% RH. The DVS plot of Type A in FIG. 44 showed 0.100 water uptake at 25° C./80VRH, indicating Type A is non-hygroscopic. As shown in FIG. 45, no XRPD pattern difference was observed for Type A before and after the DVS test, indicating no form change. DSC curve in FIG. 46 showed only the melting endotherm of Type A at 95.0° C. (onset) after the DVS test.

### Example 17: Preparation of Type C of N-(3,5-difluorobenzyl)-N-hydroxy-2,2-dimethylbutanamide

Type C of N-(3,5-difluorobenzyl)-N-hydroxy-2,2-dimethylbutanamide (817506-10-A10) was obtained via liquid vapor diffusion of a DMSO solution of a starting material (817506-01-A) in H₂O atmosphere. As shown in FIG. 47, XRPD pattern of Type C is similar to Type A, except several peaks shift (marked by dashed lines). After vacuum dried at 50° C., Type C was converted to Type A. XRPD comparison results are shown in FIG. 48.

### Example 18: Solubility Measurement of N-(3,5-difluorobenzyl)-N-hydroxy-2,2-dimethylbutanamide

Approximate solubility of N-(3,5-difluorobenzyl)-N-hydroxy-2,2-dimethylbutanamide (817506-01-A) was measured in 20 solvent systems at RT. Approximately 2 mg of the sample was added into a 3-mL glass vial. Solvents as shown in Table 36 were then added stepwise (50/50/200/700/1000 μL) into the vials until the solids were dissolved visually or a total volume of 2 mL was reached. Solubility results summarized in Table 36 were used to guide the solvent selection in preparing the solid forms as disclosed herein.

TABLE 36

| | Solubility | | Solubility |
|---|---|---|---|
| Solvent (v/v) | (mg/mL) | Solvent (v/v) | (mg/mL) |
| MeOH | S > 46.0 | 1,4-Dioxane | S > 46.0 |
| EtOH | S > 40.0 | DCM | S > 50.0 |
| IPA | S > 42.0 | CHCl₃ | S > 50.0 |
| Acetone | S > 46.0 | ACN | S > 50.0 |
| MIBK | S > 50.0 | n-Heptane | S < 1.0 |
| EtOAc | S > 44.0 | Toluene | 20.0 < S < 40.0 |
| IPAC | S > 50.0 | DMSO | S > 42.0 |
| MTBE | 2.3 < S < 7.7 | DMAc | 21.0 < S < 42.0 |
| THF | 25.0 < S < 50.0 | NMP | 2.40 < S < 48.0 |
| 2-MeTHF | 2.2 < S < 7.3 | H₂O | S < 1.3 |

### Example 19: Characterizations of Type A form of N-(3,5-difluorobenzyl)-N-hydroxy-2,2-dimethylbutanamide

Three batches of crystalline Type A form of N-(3,5-difluorobenzyl)-N-hydroxy-2,2-dimethylbutanamide were characterized using XRPD, TGA, and DSC. Results are shown in Table 37 and FIGS. 49-53. One batch of material was also characterized by NMR as shown in FIG. 54.

TABLE 37

| | | | TGA | |
|---|---|---|---|---|
| | Batch | Crystal | (%, to | DSC endotherm |
| CP ID | Number | form | 100° C.) | (° C., onset) |
| 817506-48- | SN-045-17 | Type A | 0.4 | 94.8 |
| A | | | | |
| 810048-18- | YF2022-2- | Type A | 0.4 | 95.1 |
| B | API | | | |
| | micronized | | | |
| 810055-01- | YF2022-3- | Type A | 0.9 | 94.8 |
| A | API | | | |
| | micronized | | | |

### Example 20: Characterizations of Type A form of N-(3,5-difluorobenzyl)-N-hydroxy-2,2-dimethylbutanamide Containing Impurity B

Four batches of materials comprising crystalline Type A form of N-(3,5-difluorobenzyl)-N-hydroxy-2,2-dimethylbutanamide and Impurity B were characterized using XRPD and DSC. Results are shown in Table 38 and FIGS. 55 and 56.

TABLE 38

| | | Crystal | DSC endotherm |
|---|---|---|---|
| CP ID | Batch Number | form | (° C., onset) |
| 817506-01-A | SN-023-51 | Type A + Impurity | 74.5, 90.0 |
| | | B | |
| 817506-38-A | SN-023-91 | Type A + Impurity | 79.2 |
| | | B | |
| 817506-38-B | SN-023-90 | Type A + Impurity | 74.3, 91.2* |
| | | B | |
| 810018-18-A | SN-023-51 | +Impurity B | 74.4, 91.3 |

*Peak endotherm.

### Example 21: Instruments and Methods

### XRPD

For the XRPD analysis as shown in FIGS. 5A and 6A, an X-ray powder diffractometer/Bruker D8 Advance (ADS-XRPD-001) instrument was used. The XRPD parameters used are listed in Table 39 Parameters for these XRPD tests.

TABLE 39

| | | | |
|---|---|---|---|
| X-ray Generator | Cux k-Alpha1, (λ = 1.54060 Å) | Tube Voltage (kV) | 40 |
| Tube Current (mA) | 40 | Divergence Slit (mm) | 0.60 |
| Primary Soiler Slit (deg) | 2.5 | Secondary Soiler Slit (deg) | 2.5 |
| Detector Slit (mm) | 10.50 | Antiscattering Slit(mm) | 7.10 |
| Scan axis | 2-Theta/Theta | Step size(deg) | 0.02 |
| Scanning Speed (deg/min) | 10 | Scanning Scope (deg) | 4.40 |
| Detector* | PSD: LynxEye | Use default | 2.5 |
| Scan type | Locked Coupled | Scan mode | Continuous Scan |
| Sample rotation speed | 15 rpm | Time/step(s) | 0.12 |

*Note:
The detector SSD160 belong to LynxEye.

For the XRPD analysis as described in Examples 2-20 and those as shown in the figures other than FIGS. 5A and 6A, X′pert 3 X-ray powder diffractometers were used. The testing sample was spread on the middle of a zero-background Si holder. The XRPD parameters used are listed in Table 40 Parameters for these XRPD tests.

TABLE 40

| Parameters | Reflection Mode | | Transmission M |
|---|---|---|---|
| Model | Empyrean | X′ Pert3 | X′ Pert3 |
| X-Ray wavelength | Cu, kα, | Cu, kα, | Cu, kα, |
| | Kα1 (Å): 1.540598, | Kα1 (Å): 1.540598, | Kα1 (Å): 1.540598, |
| | Kα2 (Å): 1.544426 | Kα2 (Å): 1.544426 | Kα2 (Å): 1.544426 |
| | Kα2/Kα1 intensity | Kα2/Kα1 intensity | Kα2/Kα1 intensity |
| | ratio: 0.50 | ratio: 0.50 | ratio: 0.50 |
| X-Ray tube setting | 45 kV, 40 mA | 45 kV, 40 mA | 45 kV, 40 mA |
| Divergence slit | Automatic | 1/8° | 1/2° |
| Scan mode | Continuous | Continuous | Continuous |
| Scan range (°2TH) | 3°-40° | 3°-40° | 3°-40° |
| Scan step time (s) | 33.02 | 46.67 | 31.98 |
| Step size (°2TH) | 0.0167 | 0.0263 | 0.0131 |
| Test time | ~10 min | ~5 min | ~11 min |

### TGA and DSC

TGA data were collected using a TA Q5000 and Discovery TGA 5500 TGA from TA Instruments and DSC was performed using a TA Q2000 and Discovery DSC 2500DSC from TA Instruments. Detailed parameters used are listed in Table 41.

TABLE 41

| Parameters | TGA | DSC |
|---|---|---|
| Method | Ramp | Ramp |
| Sample pan | Aluminum, open | Aluminum, crimped |
| Temperature | RT-desired temperatur | 25° C. - desired temperature |
| Heating rate | 10° C./min | 10° C./min |
| Purge gas | N₂ | N₂ |

### HPLC

An Agilent 1290 UPLC with DAD detector and Agilent 1260 HPLC with VWD detector were use and detailed chromatographic conditions for purity and solubility analysis are listed in Table 42 and Table 43.

TABLE 42

| UPLC | Agilent 1290 with DAD detector | |
|---|---|---|
| Column | Waters UPLC BEH C18, 50 × 2.1 mm, 1.7 μm | |
| Mobile phase | A: 0.05% TFA in H₂O | |
| | B: 0.05% TFA in MeOH | |
| | Time (min) | % B |
| Gradient table | 0.00 | 40 |
| | 0.50 | 40 |
| | 3.50 | 90 |
| | 3.70 | 90 |
| | 3.71 | 40 |
| | 4.00 | 40 |
| Run time | 4.0 | min |
| Post time: | 0.0 | min |
| Flow rate | 0.8 | mL/min |
| Injection volume | 3 | μL |

| | |
|---|---|
| Detector wavelength | UV at 214 nm |
| Column temperature | 40° C. |
| Sampler temperature | RT |
| Diluent | ACN |

TABLE 43

| UPLC | Agilent 1260 with VWD detector | |
|---|---|---|
| Column | Agilent Xbridge C18, 150 × 4.6 mm 5 μm | |
| Mobile phase | A: 0.05% TFA in H₂O | |
| | B: 0.05% TFA in ACN | |
| | Time (min) | % B |
| Gradient table | 0.00 | 30 |
| | 10.00 | 90 |
| | 12.0 | 90 |
| | 12.01 | 30 |
| | 15.00 | 30 |

| | |
|---|---|
| Run time | 15.0 min |
| Post time | 0.0 min |
| Flow rate | 1.0 mL/min |
| Injection volume | 5 μL |
| Detector wavelength | UV at 214 mm |
| Column temperature | 40° C. |
| Sampler temperature | RT |
| Diluent | ACN |

### DVS

DVS was measured Via an SMS (Surface Measurement Systems) DVS Intrinsic. The relative humidity at 25° C. was calibrated against deliquescence point of LiCl. Mg(NO₃)₂ and KCl. Parameters for DVS test were listed in Table 44.

TABLE 44

| Parameters | DVS |
|---|---|
| Temperature | 25° C. |
| Sample size | 20-40 mg |
| Gas and flow rate | N₂, 200 mL/min |
| dm/dt | 0.002%/min |
| Min. dm/dt stability duration | 10 min |
| Max. equilibrium time | 180 min |
| RH range | 0% RH-95% RH-0% RH |
| RH step size | 10% RH from 0% RH to |
| | 90% RH & 90% RH to 0% RH |
| | 5% RH from 90% RH to |
| | 95% RH & 95% RH to 90% RH |

### PLM

The PLM images were captured with ZEISS Scope. A1 microscope.

### ¹H NMR

¹H (proton) solution NMR was collected on a Bruker 400M NMR Spectrometer using DMSO-d6 as a solvent.

All publications, including but not limited to disclosures and disclosure applications, cited in this specification are herein incorporated by reference as though fully set forth. If certain content of a publication cited herein contradicts or is inconsistent with the present disclosure, the present disclosure controls.

One skilled in the art will readily recognize from the disclosure and claims that various changes, modifications, and variations can be made therein without departing from the spirit and scope of the disclosure as defined in the following claims.

1. Crystalline Compound N-(3,5-difluorobenzyl)-N-hydroxy-2,2-dimethylbutanamide.

2. Crystalline Type A of the crystalline Compound N-(3,5-difluorobenzyl)-N-hydroxy-2,2-dimethylbutanamide of claim 1.

3. The crystalline Type A of claim 2, wherein the crystalline Type A is substantially pure.

4. The crystalline Type A of claim 2, wherein the crystalline Type A has an XRPD pattern substantially the same as one of the XRPD patterns indicated for crystalline Type A of N-(3,5-difluorobenzyl)-N-hydroxy-2,2-dimethylbutanamide shown in FIG. 1, FIG. 5A, FIG. 6A, FIG. 7, FIG. 9, FIG. 11, FIG. 12, FIG. 13, FIG. 15, FIG. 16, FIG. 18, FIG. 20, FIG. 22, FIG. 24, FIG. 26, FIG. 29, FIG. 30, FIG. 32, FIG. 34, FIG. 40, FIG. 41, FIG. 45, FIG. 47, FIG. 48, FIG. 49, FIG. 50, FIG. 52, and FIG. 55.

5. The crystalline Type A of claim 2, wherein the crystalline Type A has an XRPD pattern comprising degree 2θ (2 theta)-peaks (±0.2 degrees 2θ) at 9.15, 17.15, 18.22, 21.8, and 27.40.

6. The crystalline Type A of claim 2, wherein the crystalline Type A has an XRPD pattern comprising degree 2θ (2 theta)-peaks (±0.2 degrees 2θ) at 13.96 and one of the degree 2θ-peaks (±0.2 degrees 2θ) at 9.15, 14.15, 17.15, 18.22, and 26.31.

7. The crystalline Type A of claim 2, wherein the crystalline Type A has an XRPD pattern comprising degree 2θ (2 theta)-peaks (±0.2 degrees 2θ) at 13.96 and two of the degree 2θ-peaks (±0.2 degrees 2θ) at 9.15, 14.15, 17.15, 18.22, and 26.31.

8. The crystalline Type A of claim 2, wherein the crystalline Type A has an XRPD pattern comprising any three of degree 2θ-peaks (±0.2 degrees 2θ) selected from the group consisting of 9.15, 13.96, 14.15, 17.15, 18.22, and 26.31.

9. The crystalline Type A of claim 2, wherein the crystalline Type A has an XRPD pattern comprising degree 2θ-peaks (±0.2 degrees 2θ) at 9.15, 13.96, 14.15, 17.15, 18.22, and 26.31.

10. The crystalline Type A of claim 2, wherein the crystalline Type A has an XRPD pattern comprising degree 2θ (2 theta)-peaks (±0.2 degrees 2θ) at 18.20 and one of the degree 2θ-peaks (±0.2 degrees 2θ) at 9.10, 17.10, 21.7, and 27.40.

11. The crystalline Type A of claim 2, wherein the crystalline Type A has an XRPD pattern comprising degree 2θ (2 theta)-peaks (±0.2 degrees 2θ) at 18.20 and two of the degree 2θ-peaks (±0.2 degrees 2θ) at 9.10, 17.10, 21.7, and 27.40.

12. The crystalline Type A of claim 2, wherein the crystalline Type A has an XRPD pattern comprising any three of degree 2θ-peaks (±0.2 degrees 2θ) selected from the group consisting of 9.10, 17.10, 18.20, 21.7, and 27.40.

13. The crystalline Type A of claim 2, wherein the crystalline Type A has an XRPD pattern comprising degree 2θ-peaks (±0.2 degrees 2θ) at 9.10, 17.10, 18.20, 21.7, and 27.40.

14. The crystalline Type A of claim 2, wherein the crystalline Type A is anhydrous.

15. The crystalline Type A of claim 2, wherein the crystalline Type A has a differential scanning calorimetry thermogram, before decomposition of the Compound, substantially the same as one of the DSC thermograms shown in FIG. 2, FIG. 5B (before decomposition of the Compound), FIG. 6B (before decomposition of the Compound), FIG. 8, FIG. 10, FIG. 14, FIG. 17, FIG. 19, FIG. 21, FIG. 23, FIG. 25, FIG. 27, FIG. 31, FIG. 35, FIG. 36, FIG. 37, FIG. 42, FIG. 43, FIG. 46, FIG. 51, FIG. 53, and FIG. 56.

16. The crystalline Type A of claim 2, wherein the crystalline Type A has an onset melting temperature of from about 94° C. to about 96° C.

17. The crystalline Type A of claim 2 having an onset melting temperature of from about 94.5° C. to about 95.5° C.

18. The crystalline Type A of claim 2 having an onset melting temperature of about 95° C.

19. The crystalline Type A of claim 2, wherein the crystalline Type A has a thermal gravimetric analysis graph substantially the same as one of the TGA graphs shown in FIG. 3, FIG. 27, FIG. 51, and FIG. 53.

20. A process for making the crystalline Type A of N-(3,5-difluorobenzyl)-N-hydroxy-2,2-dimethylbutanamide of claim 2, comprising:
(a) dissolving N-(3,5-difluorobenzyl)-N-hydroxy-2,2-dimethylbutanamide in a first solvent to obtain a solution;
(b) adding a second solvent into the solution to obtain a suspension while stirring the solution;
(c) isolating a solid from the suspension; and
(d) drying the solid to provide crystalline Type A.

21. The process of claim 20, wherein the first solvent is a solvent in which N-(3,5-difluorobenzyl)-N-hydroxy-2,2-dimethylbutanamide has a solubility of >10 mg/ml; and the second solvent is a solvent in which N-(3,5-difluorobenzyl)-N-hydroxy-2,2-dimethylbutanamide has a solubility of <10 mg/ml.

22. The process of claim 20, wherein the first solvent is selected from the group consisting of isoamyl alcohol, ethyl lactate, MEK, anisole, n-butanol/n-BuOH, ethyl formate, 2,2,2-trifluoroethanol, toluene, pyridine, isobutanol, chlorobenzene, CPME, m-xylene, n-butyl acetate, cumene, NMP, MTBE, 2-MeTHF, EtOAc, acetone, THF, DMAc, IPA, EtOH, DCM, MeOH, IPA, MIBK, IPAc, THF, 1,4-dioxane, DCM, CHCl₃, toluene, DMSO, DMAc, NMP, and ACN; and the second solvent is selected from the group consisting of n-hexane, water, 2-Me THF, MTBE, cyclohexane, and n-heptane.

23. A process for making the crystalline Type A of N-(3,5-difluorobenzyl)-N-hydroxy-2,2-dimethylbutanamide of claim 2, which process is selected from the group consisting of anti-solvent addition, reverse anti-solvent addition, solid vapor diffusion, slurry at RT, slurry at about 50° C., slurry at about −20° C., slurry with temperature cycling, slow evaporation at RT, slow evaporation at about 20° C., fast evaporation, slow cooling, fast cooling, liquid vapor diffusion, grinding, polymer-induced method, polymer/ionic liquid induced crystallization and co-crystal screening.

24. Crystalline Type C of the crystalline Compound N-(3,5-difluorobenzyl)-N-hydroxy-2,2-dimethylbutanamide of claim 1.

25. The crystalline Type C of claim 24, wherein the crystalline Type C is substantially pure.

26. The crystalline Type C of claim 24, wherein the crystalline Type C has an XRPD pattern substantially the same as the XRPD pattern indicated for Type C shown in FIG. 47.

27. The crystalline Type C of claim 24, wherein the crystalline Type C has an XRPD pattern comprising degree 2θ (2 theta)-peaks (±0.2 degrees 2θ) at 9.08, 18.18, 21.78, and 27.39.

28. The crystalline Type C of claim 24, wherein the crystalline Type C has an XRPD pattern comprising degree 2θ (2 theta)-peaks (±0.2 degrees 2θ) at 9.08, 13.45, 14.28, 18.18, 21.78, and 27.39.

29. A composition comprising the crystalline Compound N-(3,5-difluorobenzyl)-N-hydroxy-2,2-dimethylbutanamide according to claim 1 and a pharmaceutically acceptable carrier.

30. A method of treating a disease or condition, comprising administering to a subject, a therapeutically effective amount of the crystalline Compound according to claim 1 or a pharmaceutical composition comprising the crystalline Compound according to claim 1; wherein the disease or condition is selected from a inflammatory disease, an immune disease, an allergic disease, transplant rejection, a necrotic cell disease, a neurodegenerative disease, a central nervous system (CNS) disease, ischemic brain injury, an ocular disease, an infectious disease, a malignancy, ulcerative colitis, Crohn's disease, psoriasis, rheumatoid arthritis, amyotrophic lateral sclerosis (ALS), Alzheimer's disease, and a viral infection.

31. The method according to claim 30, wherein the disease or condition is mediated by receptor-interacting protein 1 (RIP1) signalling.

32. A method of treating a disease or condition mediated by receptor-interacting protein 1 (RIP1) signalling, comprising administering to a subject, a therapeutically effective amount of the crystalline Compound according to claim 1 or a pharmaceutical composition comprising the crystalline Compound according to claim 1.

33. A method of inhibiting receptor-interacting protein 1 (RIP1), comprising contacting the RIP1 protein or a fragment thereof with the crystalline Compound according to claim 1 or a pharmaceutical composition comprising the crystalline Compound according to claim 1.
